(19) United States
(12) Patent Application Publication
(10) Pub. No.: US 2025/0145982 A1
(43) Pub. Date: May 8, 2025

(54) SYSTEM AND METHOD FOR PREPARING A SEQUENCING DEVICE

(71) Applicant: Life Technologies Corporation, Carlsbad, CA (US)

(72) Inventors: Hua Yu, Durham, NC (US); Xiaoling Yang, Madison, CT (US); Rui Zheng, Madison, CT (US); Chiu Tai Andrew Wong, Orange, CT (US); Jeremy Gray, Larkspur, CA (US)

(21) Appl. No.: 19/020,341

(22) Filed: Jan. 14, 2025

Related U.S. Application Data
(63) Continuation of application No. 16/543,389, filed on Aug. 16, 2019.
(60) Provisional application No. 62/885,668, filed on Aug. 12, 2019.
(60) Provisional application No. 62/719,081, filed on Aug. 16, 2018.
(60) Provisional application No. 62/719,078, filed on Aug. 16, 2018.

(51) Int. Cl. C12N 15/10; C12Q 1/6844; G01N 33/543

(52) U.S. Cl. CPC C12N 15/1013; C12Q 1/6844; G01N 33/54333; B01J 2219/00468; B01J 2219/005

(57) ABSTRACT

The disclosure generally relates to systems, methods, and apparatuses for magnetic bead loading. An example embodiment of the disclosure relates to an apparatus including a vertically oriented plate having a first major surface and a second major surface opposite the first major surface; a magnet holder securing a magnet in proximity to the first major surface of the vertically oriented plate; a drive mechanism coupled to the magnet holder and operable to move the magnet holder and magnet in parallel to the first major surface of the vertically oriented plate; and a substrate holder to receive a substrate and hold the substrate in a vertical orientation against the surface of the vertically oriented plate.

### CROSS-REFERENCE TO RELATED APPLICATION(S)

This application is a continuation of U.S. patent application Ser. No. 16/543,389, filed Aug. 16, 2019, which claims benefit of U.S. Provisional Application No. 62/719,081, filed Aug. 16, 2018, claims benefit of U.S. Provisional Application No. 62/719,078, filed Aug. 16, 2018, and claims benefit of U.S. Provisional Application No. 62/885,668, filed Aug. 12, 2019, each of which are incorporated herein by reference in its entirety.

### BACKGROUND

Increasingly, biological and medical research is turning to sequencing for enhancing biological studies and medicine. For example, biologist and zoologist are turning to sequencing to study the migration of animals, the evolution of species, and the origins of traits. The medical community is turning to sequencing for studying the origins of disease, sensitivity to medicines, and the origins of infection. But sequencing has historically been an expensive process, thus limiting its practice.

Among other issues, there is a challenge in loading beads modified with nucleic acid molecules into confined regions or receptacles, such as microwells or dimples, to form an array for sequencing. Placing sequencing beads in an organized, tightly packed fashion, for example, into small microwells, can increase throughput per cycle and lower customer cost. As the density of microwells increases or as the microwell size decreases, bead loading becomes difficult, leading to many open microwells and low counts of beads in wells. Too many open microwells provides for a decreased number of base reads and thus, poor sequencing performance.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of an example sequencing system.

FIG. 2 includes an illustration of an example system including a sensor array.

FIG. 3 includes an illustration of an example sensor and associated well.

FIG. 4 includes an illustration of an example method for preparing a sequencing device.

FIG. 5, FIG. 6, and FIG. 7 illustrate example schema for preparing a bead assembly.

FIG. 8 and FIG. 9 include illustrations of example bead configurations.

FIG. 10 includes a schematic presentation of an example magnetic loading system.

FIG. 11 schematically illustrates movement of a solution containing magnetic beads relative to a magnetic package at a first speed.

FIG. 12 schematically illustrates movement of a solution containing magnetic beads relative to a magnetic package at a second speed.

FIG. 13 schematically illustrates movement of a solution containing magnetic beads relative to a magnetic package in reverse direction.

FIG. 14. illustrates a microchip having beads loaded thereon.

FIG. 15 schematically illustrates a magnetic loading model.

FIG. 16, FIG. 17, FIG. 18, and FIG. 19 include illustrations of an example loading device.

FIG. 20 illustrates an example flowcell.

FIG. 21 illustrates another example flowcell having coverslips and a glass slide and moving relative to the magnets in a first direction.

FIG. 22 illustrates another example flowcell having coverslips and a glass slide and moving relative to the magnets in a second direction.

FIG. 23 includes a photo illustration of the edge of a pile within a reagent solution as it moves across an array surface.

FIG. 24 schematically represents alignment of beads to magnetic field lines.

FIG. 25 illustrates an example embodiment where the magnets are placed above the microchip.

FIG. 26 illustrates movement of bead piles relative to the magnet of the magnetic set up of FIG. 25.

The use of the same reference symbols in different drawings indicates similar or identical items.

### DETAILED DESCRIPTION

In an example, a method of preparing a sequencing device includes linking a bead support having a captured template nucleic acid modified with a linker moiety to a magnetic bead having complementary linker moiety to form a bead assembly and loading the bead assembly into a well of the sequencing device using a magnetic field. The bead assembly can be denatured to release the magnetic bead, leaving the bead support attached to a target nucleic acid in the well. The target nucleic acid can be amplified to provide a clonal population of target nucleic acids useful for sequencing the target nucleic acid.

In a further example, an apparatus includes a plate having a surface to receive a substrate having a plurality of wells, a bar magnet in proximity to a surface of the plate opposite the surface to receive the substrate, and a drive mechanism to move the bar magnet parallel to the surface of the plate. The substrate is to receive a solution including magnetic beads coupled to bead supports, e.g., sequencing beads. The magnetic beads have a greater diameter than the wells of the plurality of wells. The movement of the magnet facilitates deposition of the bead supports into the wells.

Embodiments generally relate to loading one or more sequencing beads into one or more respective microwells of an array, for example, formed on a microchip. In certain embodiments, after clonal amplification each sequencing bead can contain multiple copies of the same polynucleotide fragment.

Embodiments generally relate to systems, methods, and apparatuses for magnetic loading of bead supports. An example embodiment of the disclosure relates to mixing magnetic beads with sequencing beads to form a solution. Polynucleotides, oligonucleotides, or capture moieties can be formed on or adhered to the surface of the sequencing bead. The sequencing beads can be coupled to the magnetic beads via the polynucleotide, oligonucleotide, or capture moiety, as described below. The solution containing both beads is injected onto the surface of an array having a plurality of receptacles, such as microwells. Optionally, the magnetic beads can have larger diameter than the opening of the microwells, while the sequencing beads can have a smaller diameter to allow the sequencing beads to enter and reside in the microwell. One or more magnets positioned proximal to the microchip move back and forth parallel to the microchip surface. In an embodiment, the magnetic beads form a line and follow the movement of the magnets. During cycles of sweeping, sequencing beads load into the respective wells. The magnetic beads can be separated from the sequencing beads after the sequencing beads are loaded and can be washed away.

In a particular example, the sequencing beads include oligonucleotide probes configured to capture target polynucleotide fragments. In an example, the target polynucleotide fragment can include a capture moiety, such as a biotin, and the magnetic beads can include a complementary capture moiety, such as a streptavidin moiety, for example, described in more detail below. In another example, the oligonucleotide probe can be extended complementary to the captured target polynucleotide, the target polynucleotide can be separated from the extended oligonucleotide probe, and a further capture probe or primer complementary to a terminus of the extended oligonucleotide can be hybridized to the extended oligonucleotide. The further capture probe or primer can include the capture moiety. In a further example, capture probes complementary to the oligonucleotide probe and having the capture moiety can be hybridized to the oligonucleotide probe of the sequencing bead.

In each example, the sequencing bead and the magnetic bead can be coupled using the capture moiety and a complementary capture moiety on the surface of the magnetic bead. The sequencing beads and magnetic beads can be applied over the array. By moving one or more magnets proximal to a surface of the array, the magnetic beads are drawn across the surface and the sequencing beads enter microwells of the array. The capture moiety and complementary capture moiety can be uncoupled, separating the magnetic beads from the sequencing beads, and the magnetic beads can be washed from the surface, leaving the sequencing beads in the microwells. For example, the sequencing beads can be uncoupled from the magnetic beads by melting or chemically separating hybridized species, releasing the oligonucleotide probes of the sequencing beads from complements bound to the magnetic beads. In another example, the link between the capture moiety and the complementary capture moiety can be severed.

In a further example, the sequencing bead characteristic diameter can be selected or manipulated to be smaller than the microwell sizes, and the magnetic bead can be sized to be larger than the microwell sizes to thereby allow entry of the sequencing beads and exclude the magnetic beads. The loading process may be aided by using one or more magnets whose magnetic flux sweeps the bead mixture across the surface of the microwell.

In an example, the sequencing beads can be subjected to clonal amplification of target polynucleotides prior to coupling the magnetic bead. In another example, the sequencing beads can be subjected to clonal amplification of target polynucleotides after coupling the magnetic bead. In a further example, sequencing beads can be subjected to clonal amplification of target polynucleotides after deposition into a receptacle, such as a microwell, and after uncoupling the magnetic bead.

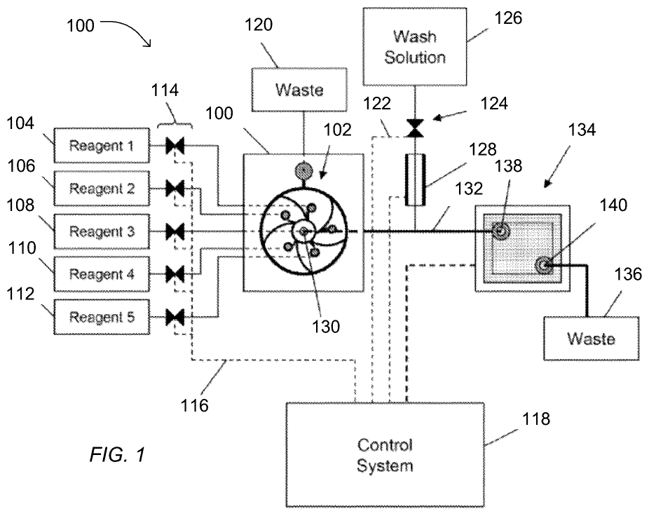

In a particular example, the loading technique can be used in a system for sequencing. Example, systems include optical sequencing systems or ion-based sequencing systems. The sequencing system can utilize optical detection of incorporated nucleotides. In another example, an ion-based sequencing system is a pH-based sequencing system utilizing a sensor substrate having microwells disposed therein. For example, FIG. 1 diagrammatically illustrates a system for carrying out pH-based nucleic acid sequencing. Each electronic sensor of the apparatus generates an output signal that depends on the value of a reference voltage. The fluid circuit permits multiple reagents to be delivered to the reaction chambers.

In an example, once the bead supports (e.g., sequencing beads) are deposited into wells and separated from the magnetic beads, the bead supports can be used in sequencing reactions. For example, the sequencing beads can include oligonucleotide portions complementary to a target sequence. A primer can be added to hybridize to a terminus of the oligonucleotide portion and sequencing reactions can be performed in a manner that permits detection of the order of the added nucleotides. In another example, the sequencing bead can have a single copy of the oligonucleotide portion and with application of a primer the target sequence can be replicated and copied to other oligonucleotide probes on the sequencing bead, yielding clonal copies of the target sequences throughout the sequencing bead. In a further example, the sequencing beads can have oligonucleotide capture probes that can capture target polynucleotides, which can be copied across the sequencing bead to provide clonal copies of the target polynucleotide.

The above loading method finds particular use in a sequencing system relying on the detection of sequencing reactions in a well. For example, the sequencing system can detect products of the sequencing reaction, such as H+ or H3O+ ions, to determine incorporation of a nucleotide. For example, a sensor component includes an array of wells associated with a sensor array. The sensors of the sensor array can include field effect transistor (FET) sensors, such as ion sensitive field effect transistors (ISFET). In an example, the wells have a depth or thickness in a range of 100 nm to 10 micrometers. In another example, the wells can have a characteristic diameter in a range of 0.1 micrometers to 2 micrometers. The sensor component can form part of a sequencing system.

In FIG. 1, a system 100 containing fluidics circuit 102 is connected by inlets to at least two reagent reservoirs (104, 106, 108, 110, or 112), to waste reservoir 120, and to biosensor 134 by fluid pathway 132 that connects fluidics node 130 to inlet 138 of biosensor 134 for fluidic communication. Reagents from reservoirs (104, 106, 108, 110, or 112) can be driven to fluidic circuit 102 by a variety of methods including pressure, pumps, such as syringe pumps, gravity feed, and the like, and are selected by control of valves 114. Reagents from the fluidics circuit 102 can be driven through the valves 114 receiving signals from control system 118 to waste container 120. Reagents from the fluidics circuit 102 can also be driven through the biosensor 134 to the waste container 136. The control system 118 includes controllers for valves, which generate signals for opening and closing via electrical connection 116.

The control system 118 also includes controllers for other components of the system, such as wash solution valve 124 connected thereto by electrical connection 122, and reference electrode 128. Control system 118 can also include control and data acquisition functions for biosensor 134. In one mode of operation, fluidic circuit 102 delivers a sequence of selected reagents 1, 2, 3, 4, or 5 to biosensor 134 under programmed control of control system 118, such that in between selected reagent flows, fluidics circuit 102 is primed and washed, and biosensor 134 is washed. Fluids entering biosensor 134 exit through outlet 140 and are deposited in waste container 136 via control of pinch valve regulator 144. The valve 144 is in fluidic communication with the sensor fluid output 140 of the biosensor 134.

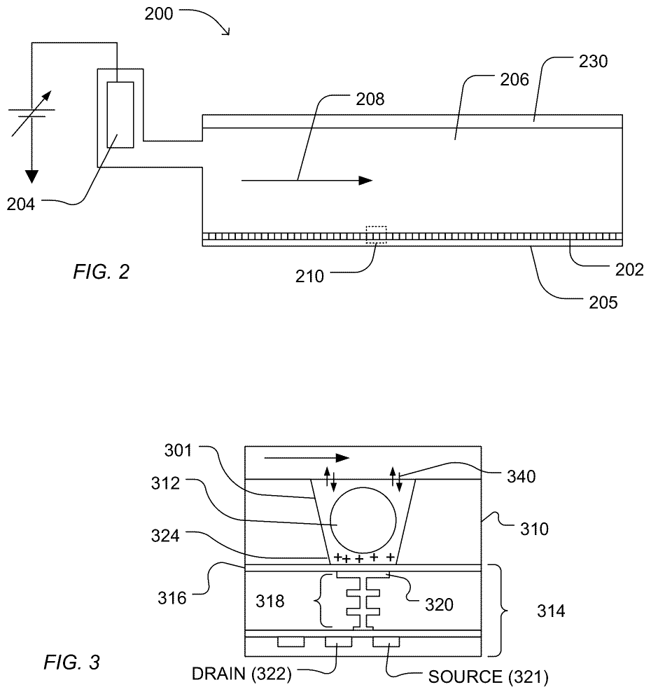

The device including the dielectric layer defining the well formed from the first access and second access and exposing a sensor pad finds particular use in detecting chemical reactions and byproducts, such as detecting the release of hydrogen ions in response to nucleotide incorporation, useful in genetic sequencing, among other applications. In a particular embodiment, a sequencing system includes a flow cell in which a sensory array is disposed, includes communication circuitry in electronic communication with the sensory array, and includes containers and fluid controls in fluidic communication with the flow cell. In an example, FIG. 2 illustrates an expanded and cross-sectional view of a flow cell 200 and illustrates a portion of a flow chamber 206. A reagent flow 208 flows across a surface of a well array 202, in which the reagent flow 208 flows over the open ends of wells of the well array 202. The well array 202 and a sensor array 205 together may form an integrated unit forming a lower wall (or floor) of flow cell 200. A reference electrode 204 may be fluidly coupled to flow chamber 206. Further, a flow cell cover 230 encapsulates flow chamber 206 to contain reagent flow 208 within a confined region.

FIG. 3 illustrates an expanded view of a well 301 and a sensor 314, as illustrated at 210 of FIG. 2. The volume, shape, aspect ratio (such as base width-to-well depth ratio), and other dimensional characteristics of the wells may be selected based on the nature of the reaction taking place, as well as the reagents, byproducts, or labeling techniques (if any) that are employed. The sensor 314 can be a chemical field-effect transistor (chemFET), more specifically an ion-sensitive FET (ISFET), with a floating gate 318 having a sensor plate 320 optionally separated from the well interior by a material layer 316. The sensor 314 can be responsive to (and generate an output signal related to) the amount of a charge 324 present on the material layer 316 opposite the sensor plate 320. The material layer 316 can be a ceramic layer, such as an oxide of zirconium, hafnium, tantalum, aluminum, or titanium, among others, or a nitride of titanium. Alternatively, the material layer 316 can be formed of a metal, such as titanium, tungsten, gold, silver, platinum, aluminum, copper, or a combination thereof. In an example, the material layer 316 can have a thickness in a range of 5 nm to 100 nm, such as a range of 10 nm to 70 nm, a range of 15 nm to 65 nm, or even a range of 20 nm to 50 nm.

While the material layer 316 is illustrated as extending beyond the bounds of the illustrated FET component, the material layer 316 can extend along the bottom of the well 301 and optionally along the walls of the well 301. The sensor 314 can be responsive to (and generate an output signal related to) the amount of a charge 324 present on the material layer 316 opposite the sensor plate 320. Changes in the charge 324 can cause changes in a current between a source 321 and a drain 322 of the chemFET. In turn, the chemFET can be used directly to provide a current-based output signal or indirectly with additional circuitry to provide a voltage-based output signal. Reactants, wash solutions, and other reagents may move in and out of the wells by a diffusion mechanism 340.

The well 301 can be defined by a wall structure, which can be formed of one or more layers of material. In an example, the wall structure can have a thickness extending from the lower surface to the upper surface of the well in a range of 0.01 micrometers to 10 micrometers, such as a range of 0.05 micrometers to 10 micrometers, a range of 0.1 micrometers to 10 micrometers, a range of 0.3 micrometers to 10 micrometers, or a range of 0.5 micrometers to 6 micrometers. In particular, the thickness can be in a range of 0.01 micrometers to 1 micrometer, such as a range of 0.05 micrometers to 0.5 micrometers, or a range of 0.05 micrometers to 0.3 micrometers. The wells 301 of array 202 can have a characteristic diameter, defined as the square root of 4 times the cross-sectional area (A) divided by Pi (e.g., sqrt (4*A/π)), of not greater than 5 micrometers, such as not greater than 3.5 micrometers, not greater than 2.0 micrometers, not greater than 1.6 micrometers, not greater than 1.0 micrometers, not greater than 0.8 micrometers or even not greater than 0.6 micrometers. In an example, the wells 301 can have a characteristic diameter of at least 0.01 micrometers. In a further example, the well 301 can define a volume in a range of 0.05 fL to 10 pL, such as a volume in a range of 0.05 fL to 1 pL, a range of 0.05 fL to 100 fL, a range of 0.05 fL to 10 fL, or even a range of 0.1 fL to 5 fL.

In an embodiment, reactions carried out in the well 301 can be analytical reactions to identify or determine characteristics or properties of an analyte of interest. Such reactions can generate directly or indirectly byproducts that affect the amount of charge adjacent to the sensor plate 320. If such byproducts are produced in small amounts or rapidly decay or react with other constituents, then multiple copies of the same analyte may be analyzed in the well 301 at the same time in order to increase the output signal generated. In an embodiment, multiple copies of an analyte may be attached to a solid phase support 312, either before or after deposition into the well 301. The solid phase support 312 may be microparticles, nanoparticles, beads, solid or porous comprising gels, or the like. For simplicity and ease of explanation, solid phase support 312 is also referred herein as a particle or bead. For a nucleic acid analyte, multiple, connected copies may be made by rolling circle amplification (RCA), exponential RCA, or like techniques, to produce an amplicon without the need of a solid support.

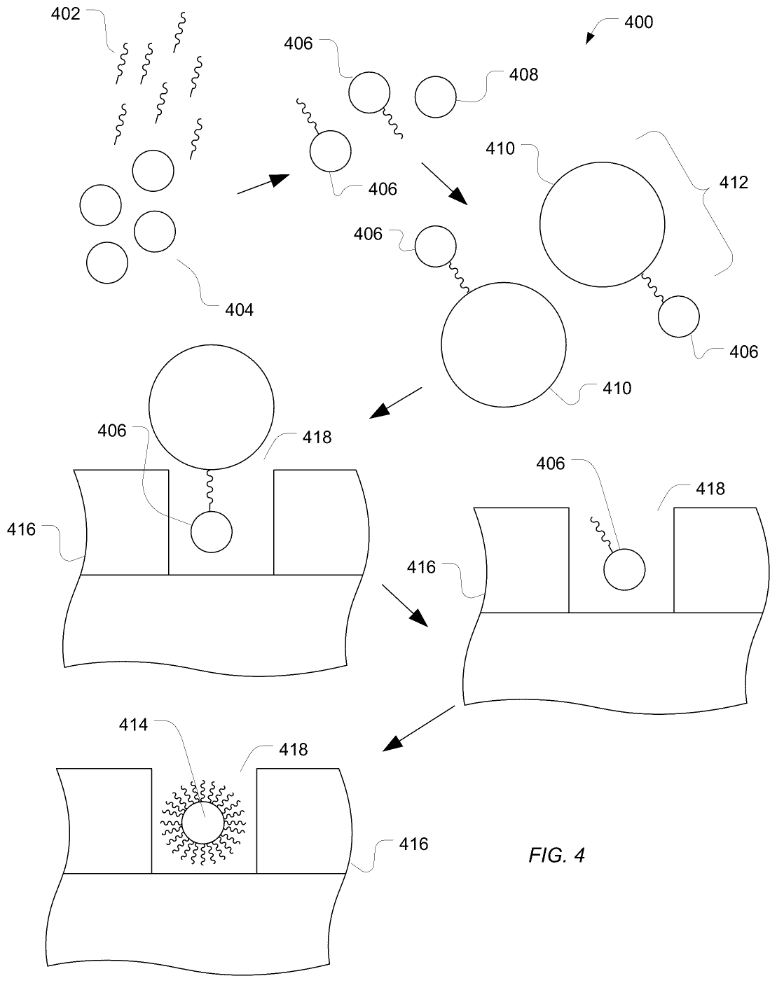

In particular, the solid phase support, such a bead support, can include copies of polynucleotides. In a particular example illustrated in FIG. 4, polymeric particles can be used as a support for polynucleotides during sequencing techniques. For example, such hydrophilic particles can immobilize a polynucleotide for sequencing using fluorescent sequencing techniques. In another example, the hydrophilic particles can immobilize a plurality of copies of a polynucleotide for sequencing using ion-sensing techniques. Alternatively, the above described treatments can improve polymer matrix bonding to a surface of a sensor array. The polymer matrices can capture analytes, such as polynucleotides for sequencing.

A bead support may be composed of organic polymers such as polystyrene, polyethylene, polypropylene, polyfluoroethylene, polyethyleneoxy, and polyacrylamide, as well as co-polymers and grafts thereof. A support may also be inorganic, such as glass, silica, controlled-pore-glass (CPG), or reverse-phase silica. The configuration of a support may be in the form of beads, spheres, particles, granules, a gel, or a surface. Supports may be porous or non-porous, and may have swelling or non-swelling characteristics. In some embodiments, a support is an Ion Sphere Particle. Example bead supports are disclosed in U.S. Pat. No. 9,243,085, titled “Hydrophilic Polymeric Particles and Methods for Making and Using Same,” and in U.S. Pat. No. 9,868,826, titled “Polymer Substrates Formed from Carboxy Functional Acrylamide,” each of which is incorporated herein by reference.

In some embodiments, the solid support is a “microparticle,” “bead,” “microbead,” etc., (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). In an example, the support is at least 0.1 microns. Microparticles or bead supports may be made of a variety of inorganic or organic materials including, but not limited to, glass (e.g., controlled pore glass), silica, zirconia, cross-linked polystyrene, polyacrylate, polymethylmethacrylate, titanium dioxide, latex, polystyrene, etc. Magnetization can facilitate collection and concentration of the microparticle-attached reagents (e.g., polynucleotides or ligases) after amplification, and can also facilitate additional steps (e.g., washes, reagent removal, etc.). In certain embodiments, a population of microparticles having different shapes sizes or colors is used. The microparticles can optionally be encoded, e.g., with quantum dots such that each microparticle or group of microparticles can be individually or uniquely identified.

Magnetic beads (e.g., Dynabeads from Dynal, Oslo, Norway) can have a size in a range of 1 micron to 100 microns, such as 2 microns to 100 microns. The magnetic beads can be formed of inorganic or organic materials including, but not limited to, glass (e.g., controlled pore glass), silica, zirconia, cross-linked polystyrene, polystyrene, or a combination thereof.

In some embodiments, a bead support is functionalized for attaching a population of first primers. In some embodiments, a bead is any size that can fit into a reaction chamber. For example, one bead can fit in a reaction chamber. In some embodiments, more than one bead fit in a reaction chamber. In some embodiments, the smallest cross-sectional length of a bead (e.g., diameter) is about 50 microns or less, or about 10 microns or less, or about 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers).

In general, the bead support can be treated to include a biomolecule, including nucleosides, nucleotides, nucleic acids (oligonucleotides and polynucleotides), polypeptides, saccharides, polysaccharides, lipids, or derivatives or analogs thereof. For example, a polymeric particle can bind or attach to a biomolecule. A terminal end or any internal portion of a biomolecule can bind or attach to a polymeric particle. A polymeric particle can bind or attach to a biomolecule using linking chemistries. A linking chemistry includes covalent or non-covalent bonds, including an ionic bond, hydrogen bond, affinity bond, dipole-dipole bond, van der Waals bond, and hydrophobic bond. A linking chemistry includes affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

As illustrated in FIG. 4, a plurality of bead supports 404 can be placed in a solution along with a plurality of polynucleotides 402 (target or template poylnucleotides). The plurality of bead supports 404 can be activated or otherwise prepared to bind with the polynucleotides 402. For example, the bead supports 404 can include an oligonucleotide (capture primer) complementary to a portion of a polynucleotide of the plurality of polynucleotides 402. In another example, the bead supports 404 can be modified with target polynucleotides 402 using techniques such as biotin-streptavidin binding.

In some embodiments, the template nucleic acid molecules (template polynucleotides or target polynucleotides) can be derived from a sample that can be from a natural or non-natural source. The nucleic acid molecules in the sample can be derived from a living organism or a cell. Any nucleic acid molecule can be used, for example, the sample can include genomic DNA covering a portion of or an entire genome, mRNA, or miRNA from the living organism or cell. In other embodiments, the template nucleic acid molecules can be synthetic or recombinant. In some embodiments, the sample contains nucleic acid molecules having substantially identical sequences or having a mixture of different sequences. Illustrative embodiments are typically performed using nucleic acid molecules that were generated within and by a living cell. Such nucleic acid molecules are typically isolated directly from a natural source such as a cell or a bodily fluid without any in vitro amplification. Accordingly, the sample nucleic acid molecules are used directly in subsequent steps. In some embodiments, the nucleic acid molecules in the sample can include two or more nucleic acid molecules with different sequences.

The methods can optionally include a target enrichment step before, during, or after the library preparation and before a pre-seeding reaction. Target nucleic acid molecules, including target loci or regions of interest, can be enriched, for example, through multiplex nucleic acid amplification or hybridization. A variety of methods can be used to perform multiplex nucleic acid amplification to generate amplicons, such as multiplex PCR, and can be used in an embodiment. Enrichment by any method can be followed by a universal amplification reaction before the template nucleic acid molecules are added to a pre-seeding reaction mixture. Any of the embodiments of the present teachings can include enriching a plurality of at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 target nucleic acid molecules, target loci, or regions of interest. In any of the disclosed embodiments, the target loci or regions of interest can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the template nucleic acid molecule. In other embodiments, the target loci or regions of interest can be between about 1 and 10,000 nucleotides in length, for example between about 2 and 5,000 nucleotides, between about 2 and 3,000 nucleotides, or between about 2 and 2,000 nucleotides in length. In any of the embodiments of the present teachings, the multiplex nucleic acid amplification can include generating at least 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 250, 300, 400, 500, 600, 700, 800, 900, 1,000, 2,000, 3,000, 4,000, 5,000, 6,000, 7,000, 8,000, 9,000, or 10,000 copies of each target nucleic acid molecule, target locus, or region of interest.

In some embodiments, after the library preparation and optional enrichment step, the library of template nucleic acid molecules can be templated onto one or more supports. The one or more supports can be templated in two reactions, a seeding reaction to generate pre-seeded solid supports and a templating reaction using the one or more pre-seeded supports to further amplify the attached template nucleic acid molecules. The pre-seeding reaction is typically an amplification reaction and can be performed using a variety of methods. For example, the pre-seeding reaction can be performed in an RPA reaction, a template walking reaction, or a PCR. In an RPA reaction, template nucleic acid molecules are amplified using a recombinase, polymerase, and optionally a recombinase accessory protein in the presence of primers and nucleotides. The recombinase and optionally the recombinase accessory protein can dissociate at least a portion of a double stranded template nucleic acid molecules to allow primers to hybridize that the polymerase can then bind to initiate replication. In some embodiments, the recombinase accessory protein can be a single-stranded binding protein (SSB) that prevents the re-hybridization of dissociated template nucleic acid molecules. Typically, RPA reactions can be performed at isothermal temperatures. In a template walking reaction, template nucleic acid molecules are amplified using a polymerase in the presence of primers and nucleotides in reaction conditions that allow at least a portion of double-stranded template nucleic acid molecules to dissociate such that primers can hybridize and the polymerase can then bind to initiate replication. In PCR, the double-stranded template nucleic acid molecules are dissociated by thermal cycling. After cooling, primers bind to complementary sequences and can be used for replication by the polymerase. In any of the aspects of the present teachings, the pre-seeding reaction can be performed in a pre-seeding reaction mixture, which is formed with the components necessary for amplification of the template nucleic acid molecules. In any of the disclosed aspects, the pre-seeding reaction mixture can include some or all of the following: a population of template nucleic acid molecules, a polymerase, one or more solid supports with a population of attached first primers, nucleotides, and a cofactor such as a divalent cation. In some embodiments, the pre-seeding reaction mixture can further include a second primer and optionally a diffusion-limiting agent. In some embodiments, the population of template nucleic acid molecules comprise template nucleic acid molecules joined to at least one adaptor sequence which can hybridize to the first or second primers. In some embodiments, the reaction mixture can form an emulsion, as in emulsion RPA or emulsion PCR. In pre-seeding reactions carried out by RPA reactions, the pre-seeding reaction mixture can include a recombinase and optionally a recombinase accessory protein. The various components of the reaction mixture are discussed in further detail herein.

In a particular embodiment of seeding, the hydrophilic particles and polynucleotides are subjected to polymerase chain reaction (PCR) amplification or recombinase polymerase amplification (RPA). In an example, the particles 404 include a capture primer complementary to a portion of the template polynucleotide 402. The template polynucleotide can hybridize to the capture primer. The capture primer can be extended to form beads 406 that include a target polynucleotide attached thereto. Other beads may remain unattached to a target nucleic acid and other template polynucleotide can be free floating in solution.

In an example, the bead support 406 including a target polynucleotide can be attached to a magnetic bead 410 to form a bead assembly 412. In particular, the magnetic bead 410 is attached to the bead support 406 by a double stranded polynucleotide linkage. In an example, a further probe including a linker moiety can hybridize to a portion of the target polynucleotide on the bead support 406. The linker moiety can be attached to a complementary linker moiety on the magnetic bead 410. In another example, the template polynucleotide used to form the target nucleic acid attached to beads 406 can include a linker moiety that attaches to the magnetic bead 410. In another example, the template polynucleotide complementary to target polynucleotide attached to the bead support 406 can be generated from a primer that is modified with a linker that attaches to the magnetic bead 410.

The linker moiety attached to the polynucleotide and the linker moiety attached to the magnetic bead can be complementary to and attach to each other. In an example, the linker moieties have affinity and can include: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement. In a particular example, the linker moiety attached to the polynucleotide includes biotin and the linker moiety attached to the magnetic bead includes streptavidin.

The bead assemblies 412 can be applied over a substrate 416 of a sequencing device that includes wells 418. In an example, a magnetic field can be applied to the substrate 416 to draw the magnetic beads 410 of the bead assembly 412 towards the wells 418. The bead support 406 enters the well 418. For example, a magnet can be moved in parallel to a surface of the substrate 416 resulting in the deposition of the bead support 406 in the wells 418.

The bead assembly 412 can be denatured to remove the magnetic bead 410 leaving the bead support 406 in the well 418. For example, hybridized double-stranded DNA of the bead assembly 412 can be denatured using thermal cycling or ionic solutions to release the magnetic bead 410 and template polynucleotides having a linker moiety attached to the magnetic bead 410. For example, the double-stranded DNA can be treated with low ion-content aqueous solutions, such as deionized water, to denature and separate the strands. In an example, a foam wash can be used to remove the magnetic beads.

Optionally, the target polynucleotides 406 can be amplified, referred to herein as templating, while in the well 418, to provide a bead support 414 with multiple copies of the target polynucleotides. In particular, the bead 414 has a monoclonal population of target polynucleotides. Such an amplification reactions can be performed using polymerase chain reaction (PCR) amplification, recombination polymerase amplification (RPA) or a combination thereof. Alternatively, amplification can be performed prior to depositing the bead support 414 in the well.

In a particular embodiment, an enzyme such as a polymerase is present, bound to, or is in close proximity to the particles or beads. In an example, a polymerase is present in solution or in the well to facilitate duplication of the polynucleotide. A variety of nucleic acid polymerase may be used in the methods described herein. In an example embodiment, the polymerase can include an enzyme, fragment or subunit thereof, which can catalyze duplication of the polynucleotide. In another embodiment, the polymerase can be a naturally occurring polymerase, recombinant polymerase, mutant polymerase, variant polymerase, fusion or otherwise engineered polymerase, chemically modified polymerase, synthetic molecules, or analog, derivative or fragment thereof. Example enzymes, solutions, compositions, and amplification methods can be found in WO2019/094,524, titled “METHODS AND COMPOSITIONS FOR MANIPULATING NUCLEIC ACIDS”, which is incorporated herein by reference in its entirety.

While the polynucleotides of bead support 414 are illustrated as being on a surface, the polynucleotides can extend within the bead support 414. Hydrogel and hydrophilic particles having a low concentration of polymer relative to water can include polynucleotide segments on the interior of and throughout the bead support 414 or polynucleotides can reside in pores and other openings. In particular, the bead support 414 can permit diffusion of enzymes, nucleotides, primers and reaction products used to monitor the reaction. A high number of polynucleotides per particle produces a better signal.

In an example embodiment, the bead support 414 can be utilized in a sequencing device. For example, a sequencing device 416 can include an array of wells 418.

In an example, a sequencing primer can be added to the wells 418 or the bead support 414 can be pre-exposed to the primer prior to placement in the well 418. In particular, the bead support 414 can include bound sequencing primer. The sequencing primer and polynucleotide form a nucleic acid duplex including the polynucleotide (e.g., a template nucleic acid) hybridized to the sequencing primer. The nucleic acid duplex is an at least partially double-stranded polynucleotide. Enzymes and nucleotides can be provided to the well 418 to facilitate detectible reactions, such as nucleotide incorporation.

Sequencing can be performed by detecting nucleotide addition. Nucleotide addition can be detected using methods such as fluorescent emission methods or ion detection methods. For example, a set of fluorescently labeled nucleotides can be provided to the system 416 and can migrate to the well 418. Excitation energy can be also provided to the well 418. When a nucleotide is captured by a polymerase and added to the end of an extending primer, a label of the nucleotide can fluoresce, indicating which type of nucleotide is added.

In an alternative example, solutions including a single type of nucleotide can be fed sequentially. In response to nucleotide addition, the pH within the local environment of the well 418 can change. Such a change in pH can be detected by ion sensitive field effect transistors (ISFET). As such, a change in pH can be used to generate a signal indicating the order of nucleotides complementary to the polynucleotide of the particle 410.

In particular, a sequencing system can include a well, or a plurality of wells, disposed over a sensor pad of an ionic sensor, such as a field effect transistor (FET). In embodiments, a system includes one or more polymeric particles loaded into a well which is disposed over a sensor pad of an ionic sensor (e.g., FET), or one or more polymeric particles loaded into a plurality of wells which are disposed over sensor pads of ionic sensors (e.g., FET). In embodiments, an FET can be a chemFET or an ISFET. A “chemFET” or chemical field-effect transistor, includes a type of field effect transistor that acts as a chemical sensor. The chemFET has the structural analog of a MOSFET transistor, where the charge on the gate electrode is applied by a chemical process. An “ISFET” or ion-sensitive field-effect transistor, can be used for measuring ion concentrations in solution; when the ion concentration (such as H+) changes, the current through the transistor changes accordingly.

In embodiments, the FET may be a FET array. As used herein, an “array” is a planar arrangement of elements such as sensors or wells. The array may be one or two dimensional. A one-dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In embodiments, one or more microfluidic structures can be fabricated above the FET sensor array to provide for containment or confinement of a biological or chemical reaction. For example, in one implementation, the microfluidic structure(s) can be configured as one or more wells (or wells, or reaction chambers, or reaction wells, as the terms are used interchangeably herein) disposed above one or more sensors of the array, such that the one or more sensors over which a given well is disposed detect and measure analyte presence, level, or concentration in the given well. In embodiments, there can be a 1:1 correspondence of FET sensors and reaction wells.

Returning to FIG. 4, in another example, a well 418 of the array of wells can be operatively connected to measuring devices. For example, for fluorescent emission methods, a well 418 can be operatively coupled to a light detection device. In the case of ionic detection, the lower surface of the well 418 may be disposed over a sensor pad of an ionic sensor, such as a field effect transistor.

One example system involving sequencing via detection of ionic byproducts of nucleotide incorporation is the Ion Torrent PGM™, Proton™ or S5™ sequencer (Thermo Fisher Scientific), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™, Proton™, or S5™ sequencer detects the nucleotide incorporations by detecting the hydrogen ion byproducts of the nucleotide incorporations. The Ion Torrent PGM™, Proton™ or S5™ sequencer can include a plurality of template polynucleotides to be sequenced, each template disposed within a respective sequencing reaction well in an array. The wells of the array can each be coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor can provide output signals indicative of nucleotide incorporation which can be represented as voltage changes whose magnitude correlates with the H+ ion concentration in a respective well or reaction chamber. Different nucleotide types can be flowed serially into the reaction chamber and can be incorporated by the polymerase into an extending primer (or polymerization site) in an order determined by the sequence of the template. Each nucleotide incorporation can be accompanied by the release of H+ ions in the reaction well, along with a concomitant change in the localized pH. The release of H+ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously.

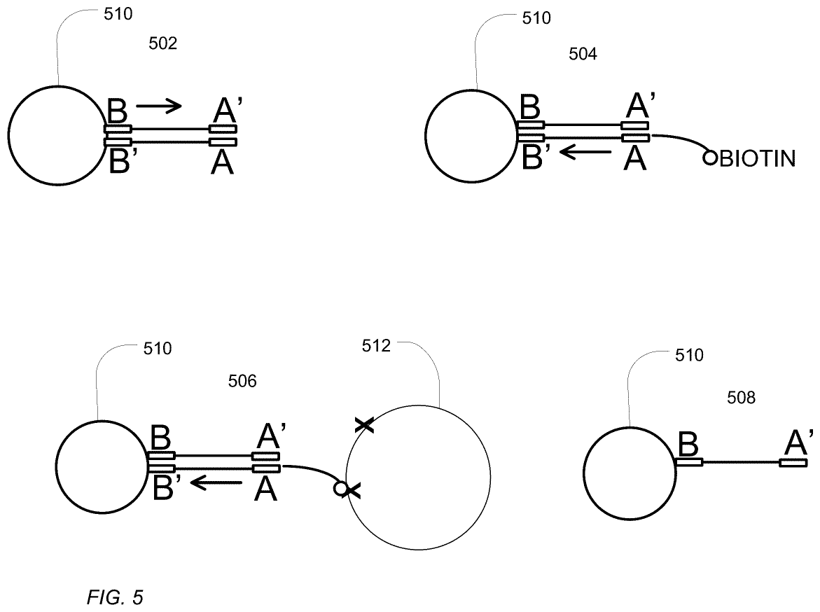

Seeding the bead supports and capture by the magnetic beads can be performed through various methods. For example, turning to FIG. 5 at 502, a template polynucleotide (B′-A) can be captured by a capture probe (B) attached to a bead support 510. The capture probe (B) can be extended complementary to the template polynucleotide. Optionally, the resultant double-stranded polynucleotide can be denatured removing the template nucleic acid (B′-A) and leaving a single-stranded (B-A′) attached to the bead support 510. As illustrated at 504, a primer (A) modified with a linker moiety, such as biotin, can be hybridized to a portion (A′) of the nucleic acid (B-A′) attached to the bead support 510. Optionally, the primer (A) can be extended to form a complementary nucleic acid (A-B′).

As illustrated 506, a magnetic bead 512 can be introduced to the solution. The magnetic bead 512 can include a linker complementary to the linker moiety attached to the primer (A). For example, the linker attached to the primer (A) can be biotin and the magnetic bead 512 can be coated with streptavidin. As described above, the magnetic bead 512 can be utilized to clean the solution and to assist with deposition of the bead support 510 and the attached nucleic acid (B-A′) into a well of a sequencing device. As illustrated 508, double-stranded polynucleotide can be denatured, resulting in the dehybridization of the nucleic acid (B′-A) from the nucleic acid (B-A′) attached to the bead support 510. As such, the bead support 510 is deposited into the wells of the sequencing device and has a single stranded target nucleic acid (B-A′). Alternatively, the linker modified probe (A) may not be extended to form a complementary polynucleotide with a length the polynucleotide (B-A′). Extension reactions can be carried out using polymerase chain reaction (PCR), recombinase polymerase amplification (RPA), or other amplification reactions.

In another example illustrated in FIG. 6, a target polynucleotide B-A′ and its complement, a template polynucleotide (A-B′), are amplified in the presence of a bead support having a capture primer. The target polynucleotide has a capture portion (B) the same as or substantially similar to a sequence of the capture primer coupled to the bead support. Substantially similar sequences are sequences whose complements can hybridize to each of the substantially similar sequences. The bead support can have a capture primer that is the same sequence or a sequence substantially similar to that of the B portion of the target polynucleotide to permit hybridization of the complement of the capture portion (B) of the target polynucleotide with the capture primer attached to the bead support. Optionally, the target polynucleotide can include a second primer location (P1) adjacent to the capture portion (B) of the target polynucleotide and can further include a target region adjacent the primers and bounded by complement portion (A′) to a sequencing primer portion (A) of the target polynucleotide.

When amplified in the presence of the bead support including a capture primer, the template polynucleotide complementary to the target polynucleotide can hybridize with the capture primer (B). The target polynucleotide can remain in solution. The system cannot undergo an extension in which the capture primer B is extended complementary to the template polynucleotide yielding a target sequence bound to the bead support.

A further amplification can be performed in the presence of a free primer (B), the bead support, and a free modified sequencing primer (A) a having a linker moiety (L) attached thereto. The primer (B) and the modified primer (L-A) can interfere with the free-floating target polynucleotide and template polynucleotide, hindering them from binding to the bead support and each other. In particular, the modified sequencing primer (A) having the linker moiety attached thereto can hybridize with the complementary portion (A′) of the target polynucleotide attached to the bead support. Optionally, the linker modified sequencing primer L-A hybridized to the target polynucleotide can be extended forming a linker modified template polynucleotide. Such linker modified template polynucleotide hybridize to the target nucleic acid attached to the bead support can then be captured by a magnetic bead and used for magnetic loading of the sequencing device.

The amplification or extensions can be performed using polymerase chain reaction (PCR) amplification, recombinase polymerase amplification (RPA), or other amplification techniques. In a particular example, each step of the scheme illustrated in FIG. 6 is performed using PCR amplification.

In another example illustrated in FIG. 7, an alternative scheme includes a target polynucleotide (P1-A′) and its complement template polynucleotide (A-P1′). The target polynucleotide and template polynucleotide are amplified in a solution including a linker modified sequencing primer (L-A) and a truncated P1 primer (trP1) having a portion having the sequence of the capture primer (B). In an example, the truncated P1 primer (trP1) includes a subset of the sequence of P1 or all of the sequence P1. During subsequent amplifications in the presence of the linker modified sequencing primer (L-A) and truncated P1 primer (trP1-B), a single species includes a linker modified template polynucleotide (L-A-B′) operable to hybridize with a bead support having a capture primer (B). Accordingly, the linker modified template polynucleotide (L-A-B′) hybridizes with the capture primer (B) on the bead and is extended to form a target polynucleotide (B-A′) attached to the bead support.

The linker modified template polynucleotide hybridize to the target polynucleotide attached bead can be utilized to attached to a magnetic bead, which can be used to implement magnetic loading of the bead into a sequencing device. As described above, the linker moiety of the linker modified template polynucleotide can take various forms, such as biotin, which can bind to linker moieties attached to the magnetic bead, such as streptavidin. Each of the amplification reactions can be undertaken using PCR, RPA, or other amplification techniques. In the example illustrated in FIG. 7, the scheme can be implemented using three cycles of polymerase chain reaction (PCR). Such a series of PCR reactions results in a greater percentage of bead supports having a single target polynucleotide attached thereto. As a result, more monoclonal populations can be generated in wells in the sequencing device.

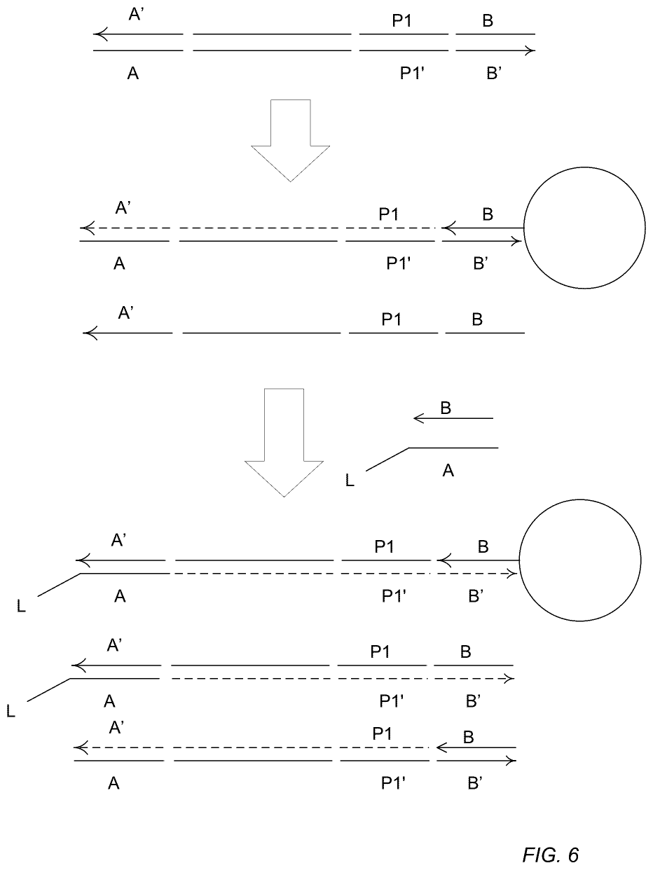

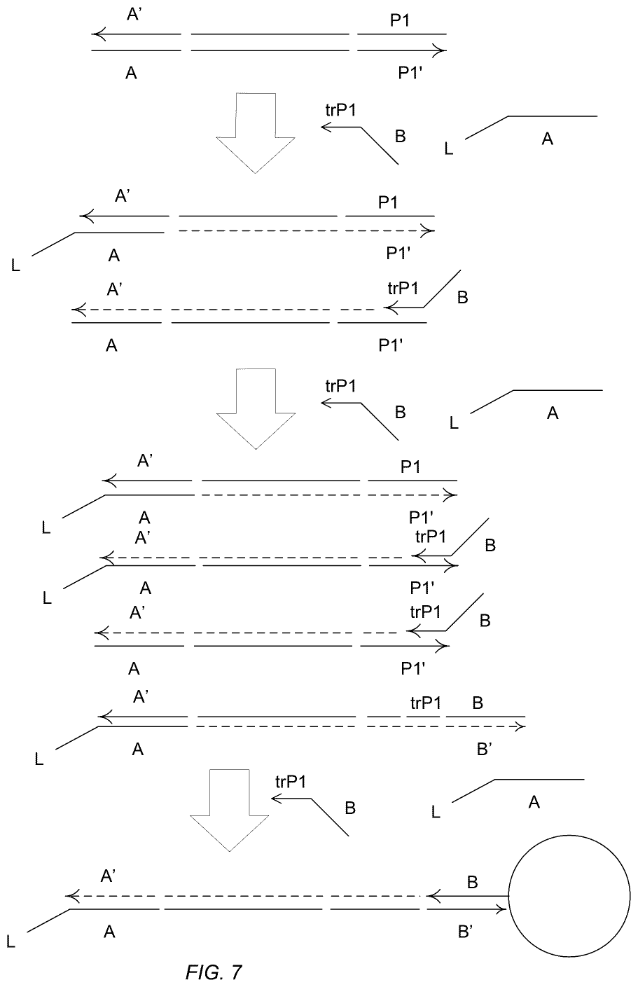

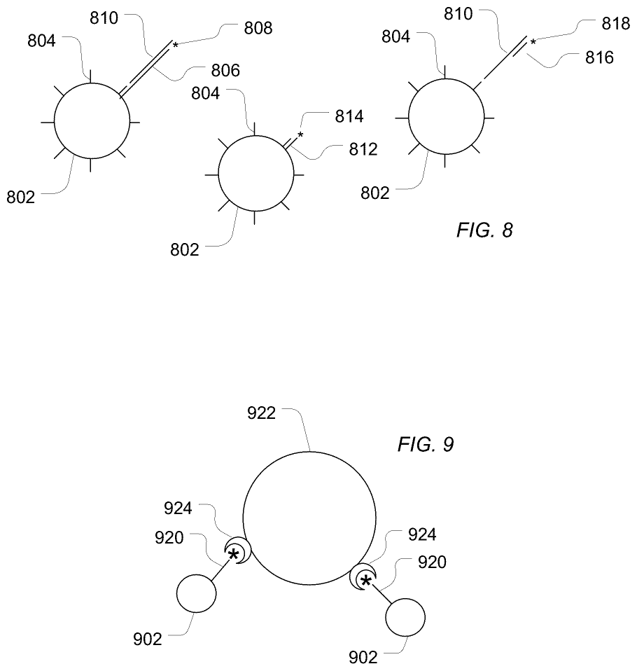

In alternative examples, as illustrated in FIG. 8, sequencing beads 802 can include exposed oligonucleotide probes 804. Such oligonucleotide probes 804 can capture target polynucleotides 806. The polynucleotide 806 can include a capture moiety 808 that is complementary to surface functionality on the magnetic beads. Optionally, the oligonucleotide probe 804 can be extended to form a portion 810 complementary to the target polynucleotide 806. In another example, the polynucleotide 806 can be stripped from the oligonucleotide probe 804 and optional portion 810 and a primer or probe 816 having a capture moiety 818 can be hybridized to a terminus of the portion 810. In another example, a capture primer 812 that includes a capture moiety 814 is configured to be captured by the oligonucleotide probe 804. As illustrated in FIG. 9, the magnetic beads 922 can include surface moieties 924 complementary to the capture moiety 920 of the sequencing beads 902. Following deposition of the sequencing beads into wells, the species (polynucleotide or primer) can be melted or otherwise detached from the oligonucleotide probe 804 or the portion 810, freeing the sequencing bead from the magnetic bead.

Capture moieties can be one of binding partners having affinity between binding partners, for example between: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; or an oligonucleotide or polynucleotide and its corresponding complement.

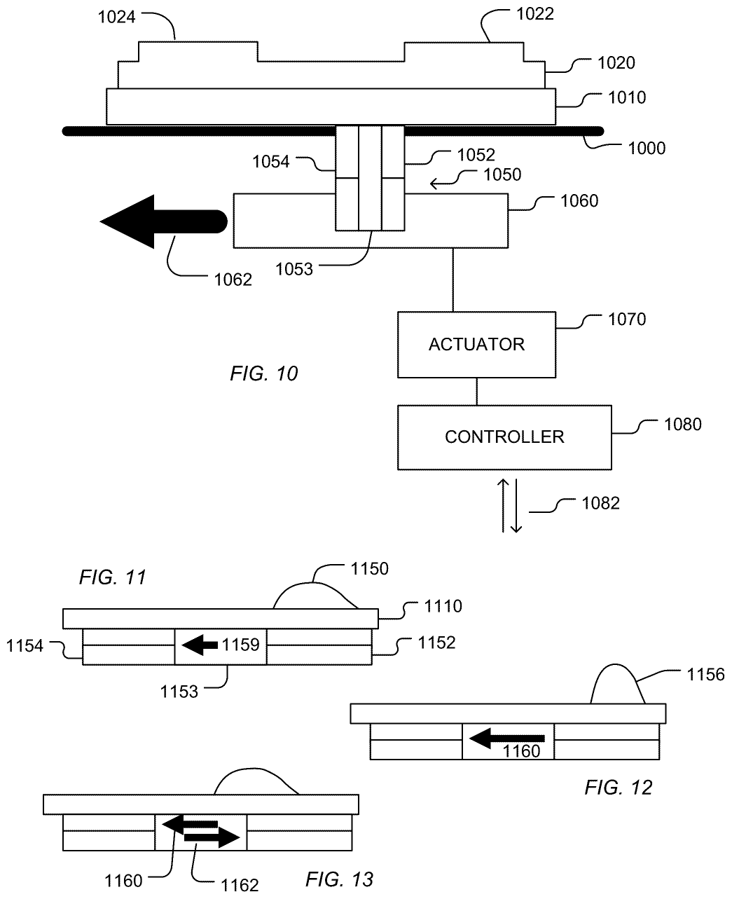

FIG. 10 is a schematic presentation of an example magnetic loading system. Specifically, FIG. 10 shows substrate 1000 supporting chip surface 1010 and flowcell 1020. Magnetic package 1050 is arranged in tray 1060 proximal to substrate 1000.

Magnetic package 1050 is shown with two magnets 1052 and 1054. Although the embodiment of FIG. 10 shows magnets 1052 and 1054, the disclosed principles are not limited thereto and may include more or less magnets than shown in FIG. 10. Magnets 1052, 1054 may be separated with an inert material 1053. The inert material 1053 can act a non-conductive insulator. In certain embodiments, magnets 1052 and 1054 can be arranged such that the north pole of magnet 1052 is immediately across the south pole of magnet 1054. With this arrangement, substrate 1000 is simultaneously exposed to the north and the south poles of magnets 1052 and 1054. In other embodiments, magnets 1052 and 1054 may be arranged such that substrate 1000 is exposed only to the north or the south pole of the magnets.

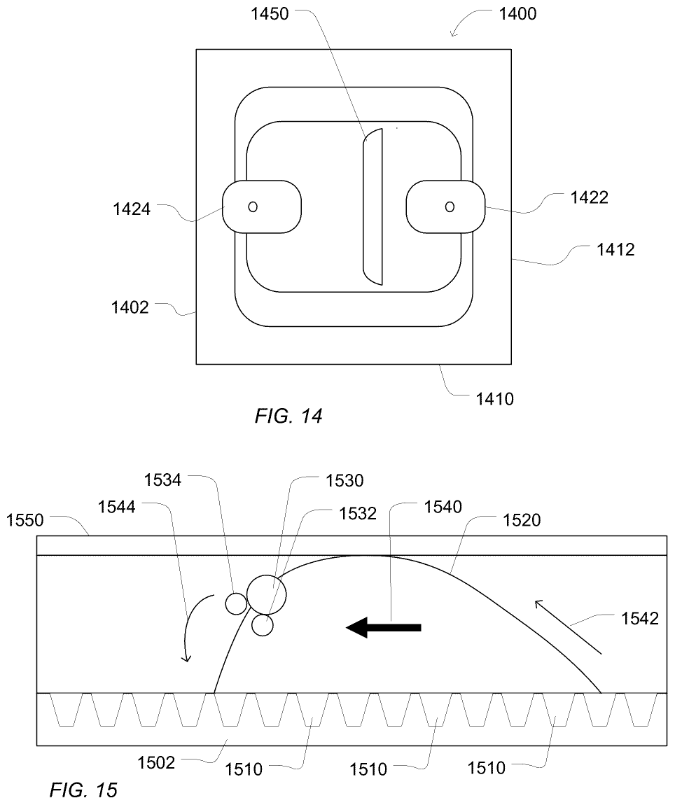

Substrate 1000 may comprise any material configured to receive microchip 1010 (interchangeably, chip). Microchip 1010 may comprise a top surface having a plurality of receptacles, such as microwells, cavities, divots, dimples or other receptacles, configured to receive one or more sequencing beads. In one embodiment, chip 1000 may comprise microwells configured to receive a sequencing bead. One such example microchip is supplied by Ion Torrent® as the Ion 541 Chip™. An example microchip is discussed below in reference to FIG. 14.

Flowcell 1020 is positioned over the upper surface of microchip 1010 to enable fluid communication to the surface of the microchip. The fluid may be communicated through ports 1022 and 1024 formed a top of chip 1010. Magnetic beads and sequencing beads (not shown) may be communicated along with one or more reagents to the surface of microchip 1010 through ports 1022 and 1024. Once the sequencing beads have been loaded onto the surface of microchip 1010, a wash reagent may be communicated through ports 1022 and 1024 to remove unwanted particles or reagents.

Tray 1060 (and magnetic package 1050) may move relative to substrate 1000, as indicated by arrow 1062. While the movement and orientation of the substrate are illustrated as being horizontal, in alternative examples, the substrate may be oriented vertically, and the movement may be up and down. The movement may be arranged by an actuator 1070 in combination with a programmable processor or controller 1080 that designates the speed and direction of movement for tray 1060. The actuator 1070 may include, for example, a motor or a solenoid controlled by a controller 1080 having one or more of a processor circuitry and a memory circuitry. The controller 1080 may be a programmable controller. In one embodiment of the disclosure, the controller 1080 may be configured to receive input information 1082 from auxiliary source(s) to indicate when tray 1062 should be moved relative to substrate 1000 (which may be stationary). The information 1082 may also include data related to the moving speed of tray 1060 as a function of the type of particle being loaded on to the chip. Such data may be stored at one or more memory circuitry associated with the controller 1080.

FIG. 11 schematically shows movement of a solution containing magnetic beads relative to a magnetic package at a first speed. In FIG. 11, the uppers surface of microchip 1110 is exposed to a reagent (or, solution) 1150. Reagent 1150 may include magnetic beads as well as sequencing beads. The magnetic beads may comprise any beads having an affinity or being reactive to a magnetic field. In one embodiment, the magnetic bead size is selected so as not allow it to enter into the microwell, cavity or a divot formed on the surface of the microchip. Example magnetic beads may be substantially spherical with a diameter of about 1 μm to 100 μm.

Magnets 1152 and 1154 are separated by inert material 1153 to form a magnetic package. Arrow 1159 shows the direction of movement of magnetic package 1150 relative to microchip 1110. Reagent 1150 is disposed on top of microchip 1110. Reagent 1150 may comprise one or more magnetic beads coupled to sequencing beads. Reagent 1150 may be a liquid, a gel or any material with texotropic and viscosity to move over a solid surface. A plurality of magnetic beads (not shown) may be disposed in reagent 1150 in a manner such that the magnetic beads may freely move or rotate relative to each other.

FIG. 12 schematically shows movement of a solution containing magnetic beads relative to a magnetic package at a second speed. FIG. 12 schematically shows a faster magnet motion (as shown by arrow 1160) relative to that of FIG. 12. Whereas the shape of reagent 1150 shows a relatively wider dispersion of reagent 1150 (containing magnetic beads), the shape of reagent 1156 suggest a narrower and densely packed reagent (containing magnetic beads). FIGS. 11 and 12 also show that when the relative movement is slow, the reagent/bead leading edge aligns with the lagging magnet's inner or leading edge. When the relative movement is fast, the reagent/bead pile falls behind the lagging magnet's front edge.

FIG. 13 schematically shows movement of a solution containing magnetic beads relative to a magnetic package reversing direction. Arrow 1162 shows the reversal of movement direction for the magnets. As seen in FIG. 13, when the magnets switch movement direction, the reagent/bead pile remains at the same location until picked up by the new lagging magnet's (1154) inner edge. Reversing direction on the magnets' movement may aid in loading the beads into the microwells or allow multiple sweeps of the reagent pile across the surface of the array on the microchip.

In an example, the magnet can be cycled between 5 and 50 sweeps (across and back), such as between 5 and 35 sweeps or 10 and 30 sweeps. In an example, each sweep takes 1 minute to 5 minutes, such as 1 minute to 3 minutes. Once bead supports load into wells, the bead assemblies can be denatured and the surface can be foam washed to remove the magnetic beads.

When implemented on a microchip, a suspension including the bead complexes is deposited into a flow cell over the microchip surface. FIG. 14 illustrates a microchip having magnetic beads loaded thereon according to one embodiment of the disclosure. More specifically, FIG. 14 shows microchip 1410 having flowcell 1412 positioned thereon. Flowcell 1412 includes ports 1422 and 1424 for receiving and discarding reagents. Microchip 1410 is placed over substrate 1410. One or more magnets (not shown) are placed below substrate 1410. The magnets create a magnetic field which causes a line of magnetic beads 1450 to form on the surface of microchip 1410. Movement of the magnets causes movement of line 1450 (i.e., magnetic beads) along the surface of microchip 1410. As the magnetic beads move along the surface, the sequencing beads coupled to the magnetic beads in the reagent enter wells or cavities on the surface of the microchip 1410.

FIG. 15 schematically illustrates a magnetic bead loading model. In FIG. 15, the microchip surface 1502 is shown with multiple microwells 1510. Stream 1520 contains, among others, sequencing beads 1532, 1534 attached to magnetic beads 1530. As illustrated in FIG. 15, sequencing beads 1532 and 1534 can have a smaller diameter than magnetic bead 1530. Microwells 1510 are sized so as to receive sequencing beads 1532, 1534. Each microwell 1510 may be configured to receive at least one sequencing bead 1532, 1534 and exclude magnetic beads 1530. While not shown, each microwell 1510 may be coupled to a sensing circuitry comprising one or more electrode, as well as electronic circuitry configured to detect presence of an analyte in microwell 1510. The analyte may be coupled to the sequencing bead or may be released as a result of one or more reaction inside the well. Surface 1550 schematically illustrates flowcell surface having input and output ports (not shown).

The sequencing beads may have different sizes. In one embodiment, the sequencing beads 1532, 1534 are selected such that at least one sequencing bead may enter a microwell. In other words, the sequencing bead diameters may be selected to be smaller than the microwell opening. While microwells 1510 are shown with tapered sidewalls, the claimed embodiment is not limited thereto and the microwells may have different shapes and forms without departing from the disclosed principles.

As shown, stream 1520 may comprise a plurality of beads. Magnetic beads 1530 may include magnetic properties. In certain embodiments, stream 1520 may comprise other reagents in addition to the beads. Magnetic beads 1530 may comprise Dynabeads® M-270 or Dynabeads® M-280, supplied by Thermo Fisher Scientific, having bead diameter of about 2.8 μm. Each magnetic bead 1530 may have, for example, streptavidin for coupling with biotinylated nucleic acids, antibodies, or other biotinylated ligands and targets. The magnetic beads 1530 can be attached to the sequencing beads 1532, 1534 using such a biotin/streptavidin binding.

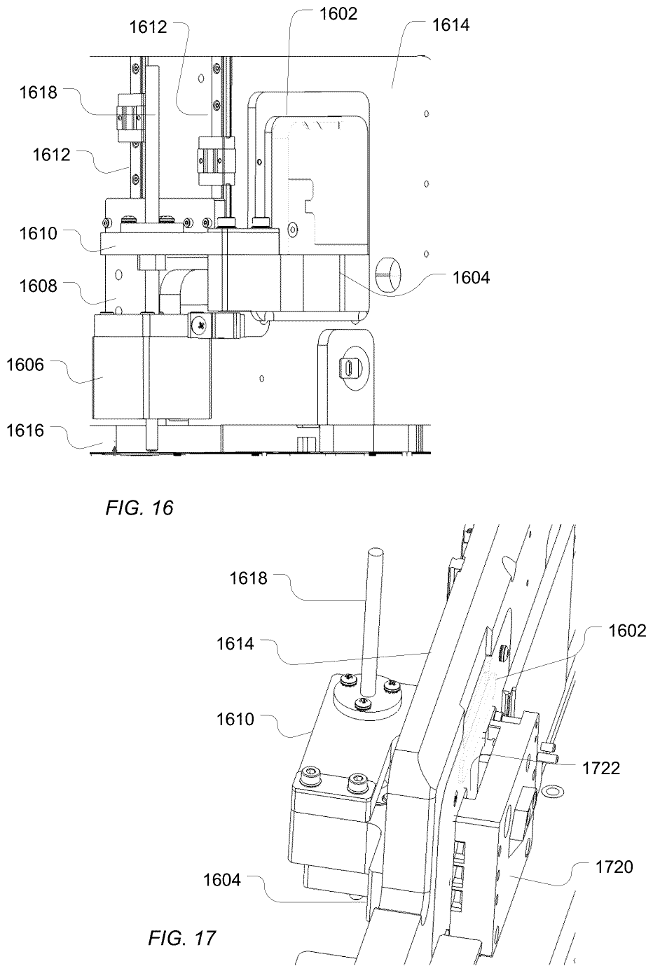

Such methods of loading may be implemented in hardware having a horizontal or vertical configuration. For example, the hardware can hold a substrate on to which beads are being deposited horizontally. In another example, the hardware can hold the substrate vertically in which the plane of the substrate approximately parallel to gravity. As used herein, vertical refers to an orientation in which a plane of a major surface of a substrate is closer to being parallel with gravity than perpendicular to gravity. In an example illustrated in FIG. 16, FIG. 17, FIG. 18, and FIG. 19, a magnetic loading system 1600 includes a plate 1602 and a magnet holder 1604 that guides magnets along the plate 1602. In the illustrated example, the plate 1602 is secured to a vertical structure 1614 that is secured to a horizontal structure 1616. The magnet holder 1604 can move magnets up and down along the plate 1602 to facilitate loading of beads supports, such as sequencing beads, into wells of a substrate disposed on opposite side of the plate 1602.

In a particular example, a drive mechanism 1606 can facilitate movement of the magnet holder 1604 up and down along the plate 1602. For example, the drive mechanism 1606 can rotate a threaded screw 1618 to drive a connector plate 1610 up and down along the screw 1618. The connector plate 1610 is connected to the magnet holder 1604. Optionally, the connector plate 1610 can be coupled with a guide plate 1608. The guide plate 1608 can slide along rails 1612, providing stability to the movement of the connector plate 1610 and the magnetic holder 1604.

As illustrated in FIG. 17, a substrate holder 1720 provides space 1722 for a substrate, such as a microchip with a flowcell, to be inserted and held against the plate 1602. As the magnets attached to the holder 1604 moved up and down along the vertical surface of the plate 1602, bead supports attached to magnetic beads in solution are deposited into wells of the substrate. In an example, the substrate is a sequencing chip having a flow cell in which the solution is disposed.

As illustrated in FIG. 18, the plate 1602 can optionally include recesses to receive heaters 1824. The heaters 1824 can be utilized to control the temperature of the plate 1602 and optionally the substrate positioned adjacent to the surface of the plate 1602. Alternatively, the heaters 1824 can be utilized to facilitate melt off of double-stranded nucleic acids.

The magnetic holder 1604 can include one or more magnets. For example, as illustrated in FIG. 19, the magnetic holder 1604 can include a magnet 1928 and a magnet 1930. The magnets 1928 or 1930 can be separated by air. Alternatively, the magnets can be separated by a paramagnetic material or insulative material.

In an example, the magnets are configured such that different polls of the magnets are positioned against the plate 1602. For example, the magnet 1928 may be configured to have a north pole positioned adjacent the plate 1602, and the magnet 1930 can be configured to have a south pole adjacent to the plate 1602. Alternatively, the south pole of the magnet 1928 and the north pole of the magnet 1930 can be positioned adjacent to the plate 1602. In a further alternative, the same pole of each magnet can be positioned adjacent the plate 1602.

The system can further include a sensor 1926 that detects a position of the magnets, for example, a lower boundary. As illustrated in FIG. 19, the guide plate 1608 can interfere with an optical sensor 1926 when the magnets are in their lower position. Alternatively, other sensors can be used to determine the position of the plates and associated magnets.

Following loading bead into wells of a microchip, polynucleotides on the sequencing beads can be amplified to form monoclonal populations of polynucleotide on the sequencing beads. The monoclonal populations of polynucleotides can be sequenced using, for example, ion-based sequencing techniques.

In the templating reaction, a sufficient number of substantially monoclonal or monoclonal populations can be produced to generate at least 100 MB, 200 MB, 300 MB, 400 MB, 500 MB, 750 MB, 1 GB or 2 GB of AQ20 sequencing reads on an Ion Torrent PGM™ 314, 316 or 318 sequencer. With respect to related high-throughput systems, a sufficient number of substantially monoclonal or monoclonal amplicons can be produced in a single amplification reaction to generate at least 100 MB, 200 MB, 300 MB, 400 MB, 500 MB, 750 MB, 1 GB, 2 GB, 5 GB, 10 GB or 15 GB of AQ20 sequencing reads on an Ion Torrent Proton, S5 or S5XL sequencer. The term “AQ20” and its variants, as used herein, refers to a particular method of measuring sequencing accuracy in the Ion Torrent PGM™ sequencer. Accuracy can be measured in terms of the Phred-like Q score, which measures accuracy on logarithmic scale that: Q10=90%, Q20-99%, Q30=99.9%, Q40=99.99%, and Q50=99.999%. For example, in a particular sequencing reaction, accuracy metrics can be calculated either through prediction algorithms or through actual alignment to a known reference genome. Predicted quality scores (“Q scores”) can be derived from algorithms that look at the inherent properties of the input signal and make fairly accurate estimates regarding if a given single base included in the sequencing “read” will align. In some embodiments, such predicted quality scores can be useful to filter and remove lower quality reads prior to downstream alignment. In some embodiments, the accuracy can be reported in terms of a Phred-like Q score that measures accuracy on logarithmic scale such that: Q10=90%, Q17=98%, Q20=99%, Q30=99.9%, Q40=99.99%, and Q50=99.999%. In some embodiments, the data obtained from a given polymerase reaction can be filtered to measure only polymerase reads measuring “N” nucleotides or longer and having a Q score that passes a certain threshold, e.g., Q10, Q17, Q100 (referred to herein as the “NQ17” score). For example, the 100Q20 score can indicate the number of reads obtained from a given reaction that are at least 100 nucleotides in length and have Q scores of Q20 (99%) or greater. Similarly, the 200Q20 score can indicate the number of reads that are at least 200 nucleotides in length and have Q scores of Q20 (99%) or greater.

In some embodiments, the accuracy can also be calculated based on proper alignment using a reference genomic sequence, referred to herein as the “raw” accuracy. This is single pass accuracy, involving measurement of the “true” per base error associated with a single read, as opposed to consensus accuracy, which measures the error rate from the consensus sequence which is the result of multiple reads. Raw accuracy measurements can be reported in terms of “AQ” scores (for aligned quality). In some embodiments, the data obtained from a given polymerase reaction can be filtered to measure only polymerase reads measuring “N” nucleotides or longer having a AQ score that passes a certain threshold, e.g., AQ10, AQ17, AQ100 (referred to herein as the “NAQ17” score). For example, the 100AQ20 score can indicate the number of reads obtained from a given polymerase reaction that are at least 100 nucleotides in length and have AQ scores of AQ20 (99%) or greater. Similarly, the 200AQ20 score can indicate the number of reads that are at least 200 nucleotides in length and have AQ scores of AQ20 (99%) or greater.

### EXAMPLES

### Example 1

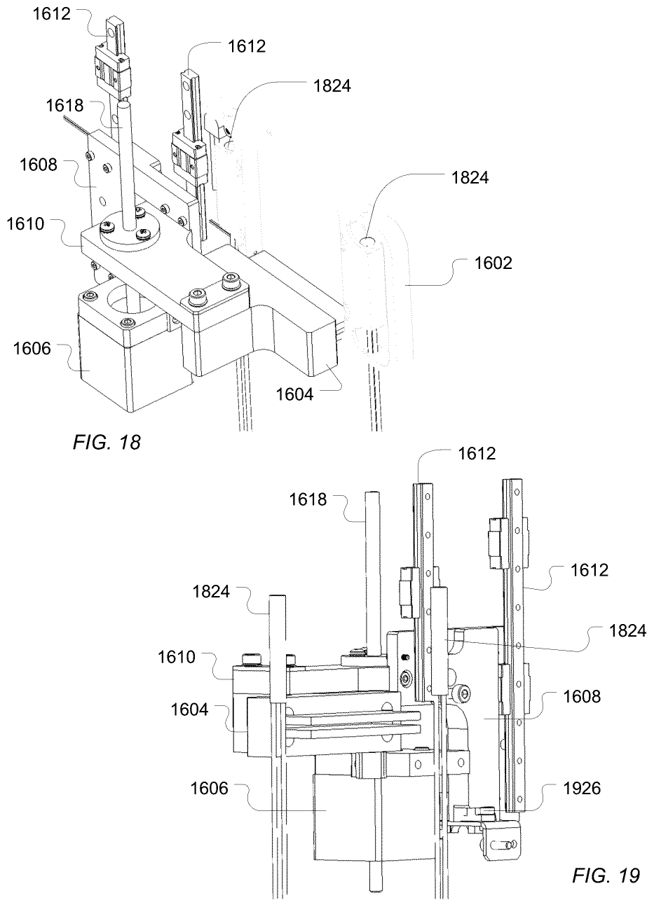

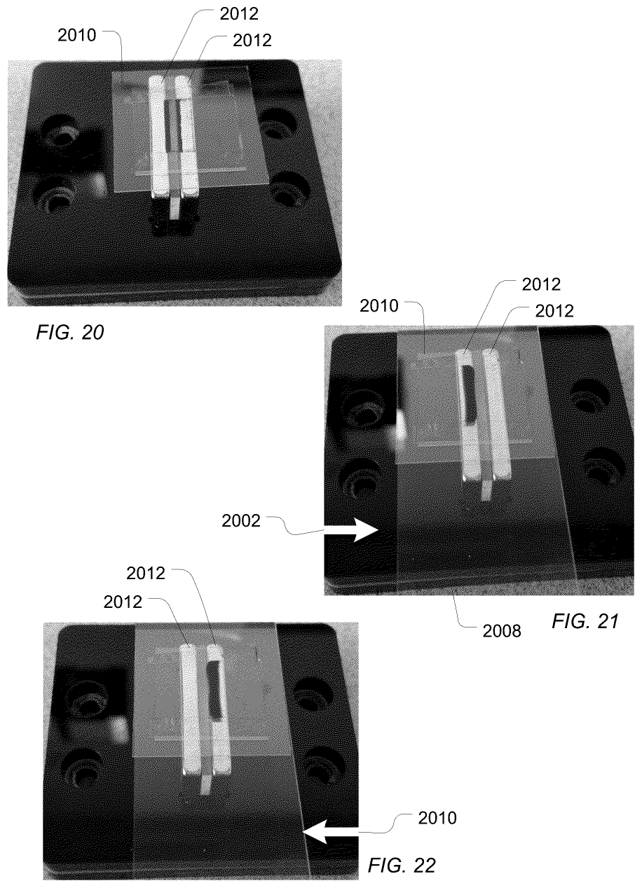

A sample apparatus is formed from coverslips to show the flow of magnetic beads within a flow cell. FIG. 20 shows an example flowcell. In FIG. 20 an example flowcell is made with coverslips 2010. The coverslips 2010 are less than about 0.2 mm thick. Double sided tape is used to bond the coverslips. The flowcell is placed directly on magnets 2012. As can be seen in FIG. 20, magnetic beads are directly attracted to the inner edges of magnets 2012.

FIG. 21 shows another example flowcell having coverslips and a glass slide and moving relative to the magnets in a first direction. In FIG. 21, flowcell 2010 and magnets 2012 were separated by glass slide 2008. Direction of movement of flowcell 2010 relative to magnets 2012 is shown by arrow 2002. It is observed that once flowcell 2010 and magnets 2012 are separated by glass slide 2008 (about 1 mm thick), the magnetic bead pile aligns with the lagging magnet as seen in FIG. 21.

FIG. 22 shows another example flowcell having coverslips and a glass slide and moving relative to the magnets in a second direction. In FIG. 22, the direction of movement is changed as shown by arrow 2010. As can be seen in FIG. 22, the microbeads now align with the lagging edge of magnets 2012.

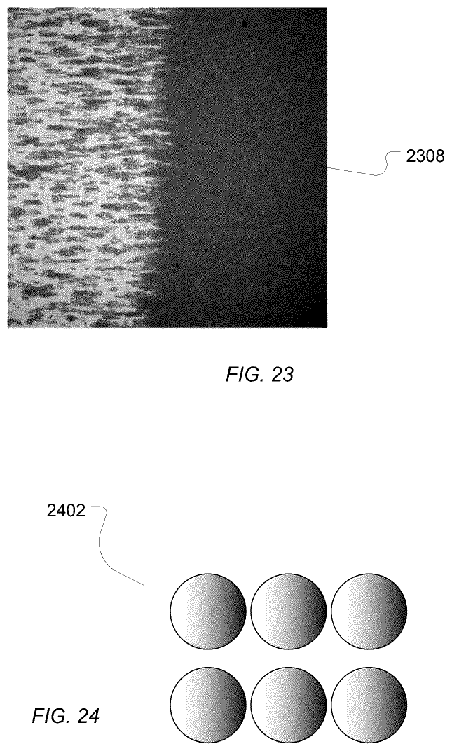

FIG. 23 shows an optically magnified image of a bead pile's leading edge. Specifically, FIG. 23 shows 20×1.6× magnification of beads on a flowcell with white light reflected. The chip is facing downwards. The flowcell is replaced with coverslip as shown in FIGS. 21 and 22. The magnets (not shown) are placed on the backside of the microchip. Beads 2308 are shown to accumulate on the right-hand side of FIG. 23. The leading front edge of the magnet (not shown) shows a distinctive rough outline.

It is believed that the magnetic beads align with the magnetic field causing attraction in the direction of the movement of the field. FIG. 24 schematically represents alignment of magnetic beads to magnetic field lines. In FIG. 24, magnetic beads 2402 are schematically shown to align with an external magnetic field (not shown). The beads' induced magnetic field causes them to attract to each other front-to-end. This attraction is schematically illustrated in the change the darker colors on the left-hand side of the bead and the light color on the right-hand side. The beads also repel each other side-by-side.

### Example 2

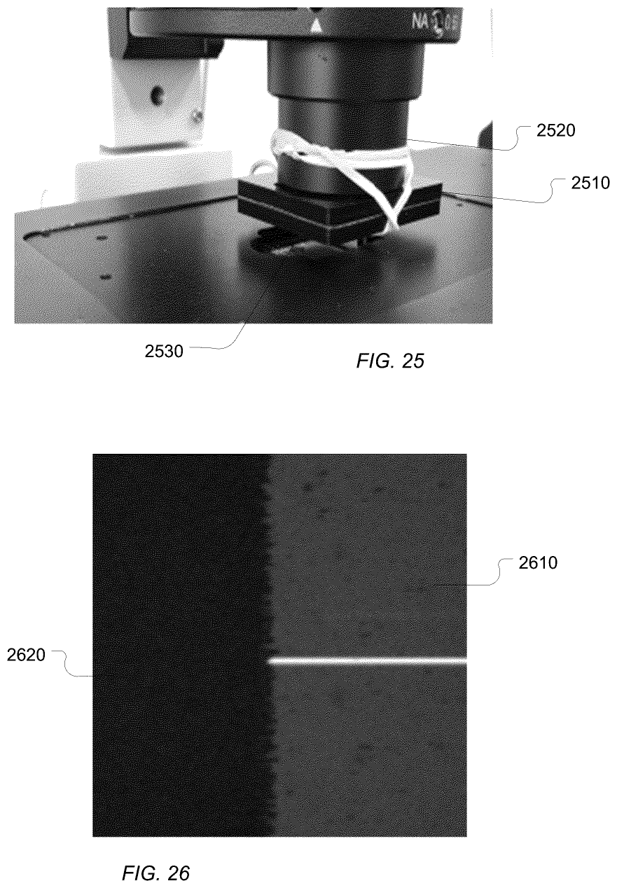

FIG. 25 shows an example embodiment where the magnets are placed above the microchip. In FIG. 25, magnets 2510 are positioned adjacent microscope objective 2520. Microchip 2530 is positioned below magnets 2510. For this experiment, magnets 2510 and objective 2520 remain stationary and the microchip is moved by automated stage.

FIG. 26 shows movement of bead piles relative to the magnet of the magnetic set up of FIG. 25. FIG. 26 shows 4×1.6× magnification. Here, the chip surface 2610 is shown relative to bead pile 2620. Rough edges can be seen to denote the microbead edges. The magnets are placed above the microchip surface 2610. The magnets remained still while the microchip is moved. White light or Cy5 fluorescence is used to obtain the image of FIG. 26.

### Example 3

A chip is loaded in accordance with the above-described methods. A second chip is loaded using a standard centrifuging technique.

For the centrifugation technique, Ion Torrent 541 chips were washed with 100 μl of 100 mM NaOH for 60 seconds, rinsed with 200 μl nuclease-free water, rinsed with 200 μl isopropyl alcohol, and aspirated dry. To load the chip, pre-seeded ISPs were vortexed, brought to 45 μl with Annealing Buffer (Ion PI™ Hi-Q™ Sequencing 200 Kit, Ion Torrent), and injected into the treated chip through the loading port.

The chip was centrifuged for 2 minutes at 1424 rcf. 1 ml of foam (980 μl 50% Annealing Buffer with 20 μl 10% Triton X-100 were combined, 1 ml of air was pipetted in, and foam was further mixed by pipette for 5 seconds) was injected into the chip and the excess was aspirated. 200 μl of a 60% Annealing Buffer/40% isopropyl alcohol flush solution was injected into the chip and the chip was aspirated to dryness. The chip was rinsed with 200 μl Annealing Buffer and the chip was vacuumed dry.

For magnetic loading, a library (2.4 B copies) was mixed with biotin TPCRA (1 uL at 100 uM) in a PCR tube. The tube is filled to 20 uL with 1× Platinum HiFi mix. The tube was thermo cycled on a thermocycler one time (2 min at 98 C, 5 min at 37 C, 5 min at 54 C). 6 billion beads were added to the tube from. 1× HiFi was added to increase volume by 50% (i.e. 20 uL of beads+10 uL of Platinum Hifi mix). The solution was thermo cycled on a thermocycler once (2 min at 98 C, 5 min at 37 C, 5 min at 54 C).

1 mL MyOne beads are pipetted into a 1.5 mL tube (1 mL MyOne beads used for 2 samples) and the tube was put on a magnet and the supernatant discarded. 1 mL 3% BSA in 1×PBS is added to the MyOne mixture, vortexed, pulse spun. The mixture was put on a magnet and the supernatant discarded. 1 mL AB is added to MyOne mixture, vortexed, pulse spun. The mixture was put on a magnet and the supernatant discarded. 250 uL AB is added to the MyOne mixture (one sample uses 125 uL 4× concentrated MyOnes). The purified MyOne mixture was transferred to new 1.5 mL tube.

Samples from the PCR tube were transfer to new 1.5 mL tube. 125 uL 4× concentrated MyOnes were added to the ISP mix. The mixture was pipetted up and down 3 times (200 uL/s) and let sit for 10 min. The mixture was put on a magnet, MyOne captured ISP were pulled out (chef speed 80 uL/s) and the supernatant was discarded. 20 uL NF water was added, pulse vortexed, pulse spun, and put on magnet to pellet MyOne.

A chip was rinsed 2× with 200 μL NF water. 20 μl of ISP mixture was mixed with 4.5 uL 10× annealing buffer and 20.5 uL water (total 45 μl). ISPs were vortexed and combined with 10× annealing buffer and water. The ISP solution was vortexes and quick spun. The ISP solution was slowly injected into the chip through the loading port. Magnetic loading was performed for 40 minutes at 30 sec/sweep. 200 μL of foam (0.2% Triton in 1×AB) was injected through the chip, and the excess is extracted. While vacuuming exit port, 200 μL 1×AB was added and then aspirated to dry chip. While vacuuming exit port, 200 μL Flush (60% AB/40% IPA) was aspirated and then aspirated to dry chip. While vacuuming exit port, 200 μL 1×AB was added.

The magnetic loaded chip demonstrates a loading of 94%, while the centrifuge loaded chip has a loading of 90%.

### Example 4

### Seeding

A library (2.4 B copies) was mixed with biotin TPCRA (1 uL at 100 uM) in a PCR tube. The tube is filled to 20 uL with 1× Platinum HiFi mix. The tube was thermo cycled on a thermocycler one time (2 min at 98 C, 5 min at 37 C, 5 min at 54 C). 6 billion beads were added to the tube from. 1× HiFi was added to increase volume by 50% (i.e. 20 uL of beads+10 uL of Platinum Hifi mix). The solution was thermo cycled on a thermocycler once (2 min at 98 C, 5 min at 37 C, 5 min at 54 C).

1 mL MyOne beads are pipetted into a 1.5 mL tube (1 mL MyOne beads used for 2 samples) and the tube was put on a magnet and the supernatant discarded. 1 mL 3% BSA in 1×PBS is added to the MyOne mixture, vortexed, pulse spun. The mixture was put on a magnet and the supernatant discarded. 1 mL AB is added to MyOne mixture, vortexed, pulse spun. The mixture was put on a magnet and the supernatant discarded. 250 uL AB is added to the MyOne mixture (one sample uses 125 uL 4× concentrated MyOnes). The purified MyOne mixture was transferred to new 1.5 mL tube.

Samples from the PCR tube were transfer to new 1.5 mL tube. 125 uL 4× concentrated MyOnes were added to the ISP mix. The mixture was pipetted up and down 3 times (200 uL/s) and let sit for 10 min. The mixture was put on a magnet, MyOne captured ISP were pulled out (chef speed 80 uL/s) and the supernatant was discarded. 20 uL NF water was added, pulse vortexed, pulse spun, and put on magnet to pellet MyOne.

### Chip Preparation

A chip was rinsed 2× with 200 μL NF water.

### Magnetic ISP Loading

20 μl of ISP mixture was mixed with 4.5 uL 10× annealing buffer and 20.5 uL water (total 45 μl). ISPs were vortexed and combined with 10× annealing buffer and water. The ISP solution was vortexes and quick spun. The ISP solution was slowly injected into the chip through the loading port. Magnetic loading was performed for 40 minutes at 30 sec/sweep. 200 μL of foam (0.2% Triton in 1×AB) was injected through the chip, and the excess is extracted. While vacuuming exit port, 200 μL 1×AB was added and then aspirated to dry chip. While vacuuming exit port, 200 μL Flush (60% AB/40% IPA) was aspirated and then aspirated to dry chip. While vacuuming exit port, 200 μL 1×AB was added. The chip is kept in 1×AB until ready to amplify ISPs on chip.

### Amplification-Keep all Reagents on Ice

### 1st Step Amplification

A tube with biotinylated primer A and blocking molecule (Neutravidin) was prepared and incubated on ice for >15 minutes. Solutions include 1.1 uL 100 uM primer per chip and 1 uL 10 mg/mL NAv (rehydrated in 0-PEG buffer) per chip. 871 μL of Rehydration buffer was added to 1× IA pellet (lot LTBP0047, PN 100032944). The solution was pulse vortexed 10×, quick spun to collect tube contents. The contents were split into two tubes of equal volume (Put 900 uL in separate tube). One tube of 900 μL was used for 1st step amplification, save other tube of 900 μL for 2nd step amplification.

For each chip to be run, 60 μL pellet solution was slowly injected into the chip. The displaced annealing buffer was aspirated from exit port. The chip was incubated with pellet solution at RT for 4 minutes. 177.4 μL start solution was added to tube of pellet solution, pulse vortexed 10× and quick spun. 110 uL/chip of starter solution was transferred to tube of primer and blocker, pulse vortexed 10× and quick spun. For each chip, ˜60 μL activated pellet solution was slowly injected into the chip. All displaced fluid was aspirated from both ports. 25 μL pellet solution was added to each port. Chips were placed onto hot plate (thermocycler) set to 40° C. The chips were covered with pipette tip box lid or similar (not the heated thermocycler cover) and let incubate for 2.5 minutes.

### Short Reaction Stop and Clean Between Amplification Steps

Amplified chips were placed near hood equipped with vacuum. While vacuuming exit port, 200 μL 0.5 M EDTA pH 8 (VWR E522-100ML) was added then aspirated to dry the chip. While vacuuming exit port, 200 μL 1×AB was aspirated and then aspirate to dry the chip. The addition of AB was repeated and the chip is left wet for 2nd step amplification. (Vac out the AB twice and leave the 3rd AB in chip)

### 2nd Step Amplification (No Blocker)

A tube with biotinylated primer A was prepared and incubated on ice for >15 minutes. Solutions include 1.1 uL 100 uM primer per chip. 871 μL of Rehydration buffer was added to 1× IA pellet (lot LTBP0047, PN 100032944). The solution was pulse vortexed 10×, quick spun to collect tube contents. After discarding appropriate volume of pellet solution, 6.6 μL 100 uM biotinylated primer was added to pellet mix and it was pulse vortexed 10×.

177.4 μL start solution was added to tube of pellet solution, pulse vortex 10× and quick spin. For each chip, ˜60 μL activated pellet solution was injected into the pre-spun chip. Displaced fluid was aspirated from both ports. An additional 25 μL pellet solution was added to each port. Chips were placed onto hot plate (thermocycler) set to 40° C. The chips were covered with pipette tip box lid or similar (not the heated thermocycler cover) and let incubate for 20 minutes.

### Reaction Stop and Clean up

Amplified chips were placed near hood equipped with vacuum. While vacuuming exit port, 200 μL 0.5 M EDTA pH 8 was added and the chips are aspirated to dry chip. While vacuuming exit port, 200 μL 1×AB) was added and then aspirated to dry chip. While vacuuming exit port, 200 μL 1% SDS solution in water (Ambion PN AM9822) was added and then aspirated to dry chip. The SDS wash is repeated. While vacuuming exit port, 200 μL formamide was added. The chip was incubated 3 minutes at 50 C, then aspirated to dry the chip. While vacuuming exit port, 200 μL Flush (50% IPA/50% AB) solution was added. The chip was aspirated to dry. While vacuuming exit port, 200 μL annealing buffer was added. The chip was left in 1×AB until ready for priming.

### On Chip Sequencing Primer Hybridization and Enzyme

Sequencing primer tube was thawed. Primer mix of final 50%/50% AB/primer mixture was prepared and vortexed well. If tube of sequencing primer has a volume of 250 μL, 250 μL 1× AB was added. The chip was aspirated to dry then 80 μL primer mix was added to the chip (50 μL in flow cell, 30 μL in ports). The chip was placed on thermocycler & incubated at 50° C. for 2 min, 20° C. for 5 min. 200 μL 1×AB was injected while vacuuming exit port. The enzyme mix was prepared with 60 μL annealing buffer & 6 μL PSP4 enzyme. The ports were cleaned and vacuumed to dry chip from the inlet port. 60 μL enzyme mix was added to the chip and incubated at RT for 5 minutes. The chip was aspirated to dry the chip from the inlet port. 100 μL of 1×AB was added to the chip immediately. The ports were cleaned, the back of the chip was dried, and the chip was loaded on the Proton for sequencing.

### Example 5

### Seeding

An Ampliseq Exome library (2.4 B copies) with A and B adapters was mixed with a 5′-biotinylated primer complimentary to the A adapter, TPCRA, (1 uL at 100 uM) in a PCR tube. The tube was filled to 20 uL with 1× Platinum HiFi mix containing Taq DNA polymerase high fidelity, salts, magnesium and dNTPs. The tube was thermo cycled on a thermocycler one time (2 min at 98° C., 5 min at 37° C., 5 min at 54° C.). Ion Sphere Particle (ISP) beads (6 billion), each having thousands of B primer immobilized thereto, were added to the tube. 1× HiFi was added to increase volume by 50% (i.e. 20 uL of beads+10 uL of Platinum Hifi mix). The solution was thermo cycled on a thermocycler once (2 min at 98° C., 5 min at 37° C., 5 min at 54° C.).

In an alternative method, in a PCR tube, 1.2 billion copies of Ion Ampliseq Exome library (20 μL 100 pM, with standard Ion Torrent A and P1 library adapters) was mixed with 3 μL 3 μM biotin-TPCRA (sequence 5′biotin-CCA TCT CAT CCC TGC GTG TC-3′) and 3 μL 1.5 μM B-trP1 (trP1 is a 23 mer segment of the Ion P1 adapter with sequence CCT CTC TAT GGG CAG TCG GTG AT; B is the ISP primer sequence) primers, and 9 μL Ion Ampliseq HiFi Master Mix 5×. The volume was filled up to 45 μL with 10 μL nuclease-free water. The tube was thermocycled on a thermocycler with the following temperature profile: 2 min at 98° C., 2 cycles of [15 sec at 98° C.-2 min at 58° C.], final hold at 10° C. After the thermocyling, 6 billion ISPs (75 μL 80 million/μL), and 6 μL Ion Ampliseq HiFi Master Mix 5× were added to the tube. 5 μL nuclease-free water was also added to bring up total volume to 131 μL. The solution was mixed well and the tube was returned to the thermocycler. A third cycle of amplification was performed with the following temperature profile: 2 min at 98° C., 5 min at 56° C., final hold at 10° C. After thermocycling, add 5 μL EDTA 0.5M and mix to stop the reaction.

### Enriching of the ISPs

MyOne superparamagnetic beads (1 mL) with streptavidin covalently coupled to the bead surface were pipetted into a 1.5 mL tube (1 mL MyOne beads used for 2 samples) and the tube was put on a magnet and the supernatant discarded. 1 mL 3% BSA in 1×PBS was added to the MyOne mixture which was then vortexed and pulse spun. The mixture was put on a magnet and the supernatant discarded. Annealing buffer (AB; 1 mL) was added to the MyOne mixture, vortexed and pulse spun. The mixture was put on a magnet and the supernatant discarded. AB (250 uL) was added to the MyOne mixture (one sample uses 125 uL 4× concentrated MyOnes). The purified MyOne mixture was transferred to a new 1.5 mL tube.

Samples from the PCR tube containing the ISP mix were transferred to a new 1.5 mL tube. Concentrated (4×) MyOne beads (125 uL) were added to the ISP mix. The mixture was pipetted up and down 3 times (200 uL/s) and then allowed to sit for 10 min. The mixture was put on a magnet, MyOne-captured ISPs were pulled out (chef speed 80 uL/s) and the supernatant was discarded. Nuclease-free (NF) water (20 uL) was added to the tube, which was then pulse vortexed, pulse spun, and put on magnet to pellet the MyOne beads.

In an alternative method of enriching the ISPs, 120 μL of MyOne Streptavidin C1 beads were transferred into a separate tube and the tube was placed on a magnet to pellet the magnetic beads. The supernatant was discarded and the tube was removed from the magnet. The beads were washed by resuspending in 150 μL Ion Torrent Annealing Buffer, then pelleting on a magnet. The supernatant was discarded, and the wash was repeated one more time with 150 μL Annealing Buffer. After discarding supernatant from the second wash, the washed MyOne C1 beads were resuspended with 50 μL Annealing Buffer. The whole content of the washed MyOne C1 in Annealing Buffer was transferred to the thermocycled PCR tube containing library and ISPs. The pipette volume was set to 160 μL, and the contents were mixed slowly by pipetting up and down three times at 1 sec per aspiration or dispensing motion. The mixture was allowed to sit at room temperature for 30 min without agitation to allow magnetic beads to capture library seeded ISPs. The tube was then put on a magnet to pellet magnetic beads and the supernatant was discarded. Tween-20 (25 μL 0.1%) in water was added to the pellet. The mixture was vortexed vigorously to elute seeded ISPs from MyOne C1 beads. The tube was pulse spun then returned to magnet. The supernatant (eluent) containing seeded ISPs was collected in a fresh tube for downstream chip loading and amplification steps.

### Chip Preparation

A chip was rinsed 2× with 200 μL NF water.

Magnetic Loading of ISPs onto Chips

Several methods of preparing the ISP/library mixture and loading it onto an Ion Torrent semiconductor chip containing reaction chamber microwells were used. In one method, the ISP/Library mixture (20 μl) was mixed with 4.5 μl 10× annealing buffer and 20.5 μl water (total 45 μl). The mixture was vortexed and spun. The ISP solution was slowly injected into the chip through the loading port. Magnetic loading was performed for 40 minutes at 30 sec/sweep. A foam (200 μL) containing 0.2% Triton in 1×AB was injected through the chip, and the excess was extracted. While vacuuming the exit port of the chip, 200 μL 1×AB was injected into the chip and then aspirated to dry the chip. While vacuuming exit port, 200 μL Flush (60% AB/40% IPA) was then injected into the chip and then aspirated to dry chip. While vacuuming exit port, 200 μL 1×AB was then added by injection into the chip. The chip was kept in 1×AB until ready to amplify the nucleic acids on the ISPs on the chip.

In another method, 150 μL Dynabeads M-270 streptavidin (Thermo Fisher Scientific), which are magnetic beads with streptavidin bound to the surface thereof, were transferred to a tube which was then placed in a magnet to pellet magnetic beads. The supernatant was discarded and the tube was removed from the magnet. The following was then added to the tube containing the M-270 pelleted beads: 20 μL ISP mixture from the seeding process, 9 μL 5× Annealing Buffer, and 16 μL nuclease-free water for a total 45 μL. Alternatively, 20 μl of ISP/Library mixture was mixed with 3.2 uL 10× annealing buffer 3 uL concentrated M270 magnetic beads and 5.8 uL water for a total of 32 μl. The mixture was mixed to resuspend the M-270 pellet, and slowly injected into the chip through the loading port. A magnet placed beneath the chip was swept across the chip back and forth repeatedly to load ISPs into chip microwells. The magnetic loading sweeping was performed for 40 minutes at 30 sec/sweep. After loading, a 15 mL falcon tube containing 5 mL 1% SDS was vigorously shaken to generate a dense foam, 800 μL of which was then injected through the chip to remove magnetic beads from the chip flow cell. Flow through at the chip exit was discarded. Annealing Buffer (200 μL) was then injected through the chip, and the flow through was discarded. The chip was vacuumed dry from the chip exit. Flush (200 μL of 60% Annealing Buffer, 40% IPA) was injected through the chip which was then vacuumed dry. Annealing Buffer (200 μL) was injected to fill the chip flow cell, and the flow through was discarded at the chip exit. The chip was left filled with Annealing Buffer until ready to amplify in downstream amplification steps.

### Amplification

### First Step Amplification

For each chip being amplified, 1.1 uL biotinylated primer A (100 uM) and 1 uL blocking molecule (10 mg/mL Neutravidin rehydrated in buffer) were combined in a tube and incubated on ice for >15 minutes.

Rehydration buffer (871 μL) was added to 1× IA pellet (PN 100032944) containing reaction components for conducting recombinase-polymerase amplification (e.g., recombinase, polymerase, single-stranded binding protein, nucleotides, buffers and other ingredients) from the ION PGM™ TEMPLATE IA 500 kit. The solution was pulse vortexed 10× and quick spun to collect tube contents. The rehydrated contents (referred to as “pellet solution”, at roughly 900 μl) were kept on ice during the process.

For each Ion Torrent chip, 60 μL of rehydrated IA pellet solution was slowly injected into the chip. The displaced annealing buffer was aspirated from the exit port. The chip was incubated with rehydrated IA pellet solution at RT for 4 minutes.

For each chip being amplified, 90 uL of rehydrated IA pellet solution was transferred to a new tube. The previously prepared biotinylated primer A and neutravidin blocking molecule (2.1 uL) was added and pulse mixed. A start solution (30 μL), containing an aqueous solution of 28 mM Mg (OAc) 2, 10 mM Tris acetate and 3.75% (V/V) methyl cellulose, was added to the tube of rehydrated IA pellet solution, pulse vortexed 10× and quick spun to form an activated amplification solution in a ˜120 uL total volume. For each chip, ˜60 μL activated amplification solution was slowly injected into the chip. All displaced fluid was aspirated from both ports. Next, 25 μL of remaining activated amplification solution was added to each chip port. Chips were placed onto a hot plate (thermocycler) set to 40° C. The chips were covered with a pipette tip box lid or similar cover (not the heated thermocycler cover) and allowed to incubate for 2.5 minutes.

### Short Reaction Stop and Clean Between Amplification Steps

Amplified chips were taken off the hot plate or thermocycler. While vacuuming the exit port, 200 μL 0.5 M EDTA pH 8 (VWR E522-100ML) was injected into the chip and the chip was then aspirated to dry using a vacuum. While vacuuming the exit port, 200 μL 1×AB was injected into the chip which was then aspirated to dry. The addition of AB was repeated two more times and the chip was left filled for the 2nd step amplification. (The AB was vacuumed out twice and the third addition of AB was left in the chip.)

### Second Step Amplification (No Blocker)

For each chip, 60 uL rehydrated pellet solution was slowing injected into the chip. The displaced annealing buffer was aspirated from the exit port. The chip was incubated with pellet solution at RT for 4 minutes.

For each chip being prepared, 90 uL of rehydrated pellet solution was transferred to a fresh tube. Biotinylated Primer A (1.1 uL of 100 uM) was added and the tube pulse vortexed and spun.

Start solution (30 μL) was added to the tube containing rehydrated pellet solution and Primer A and was pulse vortexed 10× and quick spun to generate an activated amplification solution. Approximately 60 μL activated amplification solution was injected into the chip. Displaced fluid was aspirated from both ports. An additional 25 μL of remaining amplification solution was added to each port. Chips were placed onto a hot plate (thermocycler) set to 40° C. The chips were covered with a pipette tip box lid or similar cover and allowed to incubate for 20 minutes.

### Reaction Stop and Clean Up

Chips that had been subjected to amplification reactions were placed near a hood equipped with a vacuum. While vacuuming the exit port, 200 μL 0.5 M EDTA pH 8 was added and the chips were aspirated to dry the chips. While vacuuming the exit port, 200 μL 1×AB was added and then aspirated to dry the chip. While vacuuming the exit port, 200 μL 1% SDS solution in water (Ambion PN AM9822) was added and then aspirated to dry the chip. The SDS wash was repeated. While vacuuming the exit port, 200 μL formamide was added. The chip was incubated 3 minutes at 50° C., then aspirated to dry the chip. While vacuuming the exit port, 200 uL Flush (50% IPA/50% AB) solution was added. The chip was aspirated to dry. While vacuuming the exit port, 200 μL annealing buffer was added. The chip was left in 1×AB until ready for priming.

### On Chip Sequencing Primer Hybridization and Enzyme Reaction

A tube containing Ion sequencing primer (100 uM) was thawed. For each chip being sequenced, a primer mixture of 40 uL annealing buffer and 40 uL sequencing primer was prepared and vortexed well. The chip was aspirated to dry then 80 μL of primer mixture was added to the chip (50 μL in flow cell, 15 μL in each port). The chip was placed on a thermocycler and incubated at 50° C. for 2 min, 20° C. for 5 min. 200 μL 1×AB was injected while vacuuming the exit port. An enzyme mixture was prepared with 60 μL annealing buffer and 6 μL sequencing enzyme (Ion PSP4 Sequencing Polymerase). The ports were cleaned and vacuumed to dry the chip from the inlet port. Enzyme mixture (60 μL) was added to the chip and incubated at RT for 5 minutes. The chip was aspirated to dry. AB (100 μL of 1×) was injected to fill the chip immediately. The ports were cleaned, the back of the chip was dried, and the chip was loaded on the Ion Torrent Proton (Thermo Fisher Scientific) apparatus for sequencing of the library nucleic acids.

In a first embodiment, a method to load a bead support into a reaction well of a plurality of reaction wells of a substrate, each reaction well having an inlet opening at a first surface of the substrate, includes introducing a suspension having a plurality of bead complexes onto the substrate, a bead complex of the plurality of bead complexes including a magnetic bead coupled to the bead support. The method further includes moving a magnetic apparatus parallel to a second surface of the substrate, the second surface opposite the first surface, the magnetic bead drawn to the first surface, the bead support entering into the reaction well of the plurality of reaction wells. The method also includes separating the magnetic bead from the bead support and washing the magnetic bead away from the substrate.

In an example of the first embodiment, the magnetic bead has a bead diameter larger than an opening of the plurality of reaction wells and wherein the bead support has a bead diameter smaller than the opening of the plurality of reaction wells.

In another example of the first embodiment and the above examples, the magnetic apparatus comprises a pair of magnets separated by an inert material. For example, a first magnet of the pair of magnets has a north pole disposed adjacent the second surface of the substrate and the second magnet of the pair of magnets has a south pole disposed adjacent the second surface of the substrate.

In a further example of the first embodiment and the above examples, the bead support is a sequencing bead having a polynucleotide thereon. For example, the method further includes amplifying the polynucleotide to provide multiple copies of the polynucleotide on the sequencing bead. In another example, the method further includes sequencing the polynucleotide attached to the bead support in the reaction well of the substrate.

In an additional example of the first embodiment and the above examples, moving the magnetic apparatus parallel to the second surface of the substrate includes moving the magnetic apparatus in different directions parallel to the second surface of the substrate.

In another example of the first embodiment and the above examples, the bead support is coupled to a polynucleotide having a linker moiety disposed distal from the bead support, the magnetic bead having a complementary linker moiety, the bead complex formed when the linker moiety of the bead support links with the complementary linker moiety of the magnetic bead. In an example, the polynucleotide having the linker moiety is hybridized to a second polynucleotide covalently bound to the bead support, wherein separating the magnetic bead from bead support include separating the polynucleotide from the second polynucleotide. In a further example, separating the polynucleotide from the second polynucleotide includes washing with a low ionic strength aqueous solution. In another example, separating the polynucleotide from the second polynucleotide includes heating the substrate.

In a further example of the first embodiment and the above examples, the method further includes generating a template nucleic acid including a capture sequence portion, a template portion, and primer portion modified with a linker moiety; capturing the template nucleic acid on the bead support, the bead support having a plurality of capture primers complementary to the capture sequence portion of the template nucleic acid, the capture primers hybridizing to the capture sequence portion of the template nucleic acid; linking the captured template nucleic acid to a magnetic bead having second linker moiety to form the bead complex, the second linker moiety attaching to the first linker moiety. For example, the method further includes extending the capture primer complementary to the template nucleic acid to form a sequence target nucleic acid attached to the bead support. In an example, the method further includes denaturing the template nucleic acid and the sequence target nucleic acid to release the magnetic bead from the bead support. For example, denaturing includes enzymatic denaturing. In another example, denaturing includes denaturing in the presence of an ionic solution. In an additional example, the method further includes amplifying the sequence target nucleic acid to form a population of sequence target nucleic acids on the bead support in the reaction well. In a further example, amplifying include performing recombinase polymerase amplification (RPA). In another example, performing RPA includes performing RPA for a first period, washing, and performing RPA for a second period, the first period shorter than the second period. In a further example, generating includes extending a linker modified primer complementary to a target nucleic acid. In an additional example, generating comprises amplifying a target nucleic acid having a first primer portion, a target portion, and a second primer portion in the presence of a bead support having a capture primer, a linker modified first primer complementary to the first primer portion, and a second primer having a portion complementary to at least a portion of the second primer portion, the second primer having a capture primer portion ligated to the portion and complementary to the capture primer, wherein the bead support capture primer is extended to include a sequence of the target nucleic acid. For example, amplifying includes performing three polymerase chain reaction (PCR) cycles.

In a second embodiment, an apparatus includes a vertically oriented plate having a first major surface and a second major surface opposite the first major surface; a magnet holder securing a magnet in proximity to the first major surface of the vertically oriented plate; a drive mechanism coupled to the magnet holder and operable to move the magnet holder and magnet in parallel to the first major surface of the vertically oriented plate; and a substrate holder to receive a substrate and hold the substrate in a vertical orientation against the surface of the vertically oriented plate.

In an example of the second embodiment, the substrate includes a plurality of wells.

In another example of the second embodiment and the above examples, the substrate further includes a flowcell in communication with the plurality of wells.

In a further example of the second embodiment and the above examples, the magnet holder further secures a second magnet in proximity to the first major surface of the vertically oriented plate. For example, the magnet and second magnet are oriented in parallel with a space between the magnet and second magnet. In an example, the apparatus further includes a material disposed in the space between the magnet and second magnet. In another example, the magnet is configured to have a north pole in proximity to the vertically oriented plate and the second magnet is configured to have a south pole in proximity to the vertically oriented plate. In an additional example, the apparatus further includes a connector plate connecting the magnet holder and the drive mechanism. For example, the apparatus further includes a guide plate and a rail, the guide plate coupled to the connector plate and configured to slide along the rail.

In an additional example of the second embodiment and the above examples, the drive mechanism is a screw mechanism.

In another example of the second embodiment and the above examples, the apparatus further includes a sensor to sense a position of the magnet holder.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. An apparatus comprising:
a vertically oriented plate having a first major surface and a second major surface opposite the first major surface;
a magnet holder securing a magnet in proximity to the first major surface of the vertically oriented plate;
a drive mechanism coupled to the magnet holder and operable to move the magnet holder and magnet in parallel to the first major surface of the vertically oriented plate; and
a substrate holder to receive a substrate and hold the substrate in a vertical orientation against the surface of the vertically oriented plate.

2. The apparatus of claim 1, wherein the substrate includes a plurality of wells.

3. The apparatus of claim 1, wherein the substrate further includes a flowcell in communication with the plurality of wells.

4. The apparatus of claim 1, wherein the magnet holder further secures a second magnet in proximity to the first major surface of the vertically oriented plate.

5. The apparatus of claim 4, wherein the magnet and second magnet are oriented in parallel with a space between the magnet and second magnet.

6. The apparatus of claim 5, further comprising a material disposed in the space between the magnet and second magnet.

7. The apparatus of claim 4, wherein the magnet is configured to have a north pole in proximity to the vertically oriented plate and the second magnet is configured to have a south pole in proximity to the vertically oriented plate.

8. The apparatus of claim 1, further comprising a connector plate connecting the magnet holder and the drive mechanism.

9. The apparatus of claim 8, further comprising a guide plate and a rail, the guide plate coupled to the connector plate and configured to slide along the rail.

10. The apparatus of claim 1, wherein the drive mechanism is a screw mechanism.

11. The apparatus of claim 1, further comprising a sensor to sense a position of the magnet holder.

12. A method to load a bead support into a reaction well of a plurality of reaction wells of a substrate, each reaction well having an inlet opening at a first surface of the substrate, the method comprising:
introducing a suspension having a plurality of bead complexes onto the substrate, a bead complex of the plurality of bead complexes including a magnetic bead coupled to the bead support, the bead support attached to a polynucleotide, wherein the magnetic bead has a bead diameter larger than the inlet opening of the each reaction well and wherein the bead support has a bead diameter smaller than the opening of the plurality of reaction wells, wherein the bead support is a polymeric particle having a diameter in a range of 0.1 microns to 50 microns;
moving a magnetic apparatus parallel to the first surface, the magnetic bead drawn to the first surface, the bead support entering into the reaction well of the plurality of reaction wells, wherein each reaction well of the plurality of reaction wells corresponds with an ion-sensitive sensor of a sensor array formed on the substrate;
separating the magnetic bead from the bead support; and
washing the magnetic bead away from the substrate.

13. The method of claim 12, further comprising amplifying the polynucleotide to provide multiple copies of the polynucleotide on the sequencing bead.

14. The method of claim 13, further comprising sequencing the polynucleotide attached to the bead support in the reaction well of the substrate.

15. The method of claim 12, wherein the bead support is coupled to a polynucleotide having a linker moiety disposed distal from the bead support, the magnetic bead having a complementary linker moiety, the bead complex formed when the linker moiety of the bead support links with the complementary linker moiety of the magnetic bead.

16. The method of claim 15, wherein the polynucleotide having the linker moiety is hybridized to a second polynucleotide covalently bound to the bead support, wherein separating the magnetic bead from bead support include separating the polynucleotide from the second polynucleotide.

17. The method of claim 16, wherein separating the polynucleotide from the second polynucleotide includes washing with a low ionic strength aqueous solution.

18. The method of claim 16, wherein separating the polynucleotide from the second polynucleotide includes heating the substrate.

19. The method of claim 12, further comprising:
generating a template nucleic acid including a capture sequence portion, a template portion, and primer portion modified with a linker moiety;
capturing the template nucleic acid on the bead support, the bead support having a plurality of capture primers complementary to the capture sequence portion of the template nucleic acid, the capture primers hybridizing to the capture sequence portion of the template nucleic acid; and
linking the captured template nucleic acid to a magnetic bead having second linker moiety to form the bead complex, the second linker moiety attaching to the first linker moiety.

20. The method of claim 19, further comprising extending the capture primer complementary to the template nucleic acid to form a sequence target nucleic acid attached to the bead support.
